(19) United States
(12) Patent Application Publication
(10) Pub. No.: US 2026/0256488 A1
(43) Pub. Date: Sep. 3, 2026

(54) Cutting Accessory For A Surgical Cutting Instrument

(71) Applicant: Stryker European Operations Limited, Carrigtwohill, Co Cork (IE)

(72) Inventors: Jonathan Browne, Dublin (IE); Emma O'Carroll, Parteen (IE); Conor O'Shea, Innishannon (IE); Alexander Szymanski, Kilcash Clonmel (IE); Hamed Jafarzadeh, Cork (IE); Nuala Nicolasi, Kilworth (IE); Branko Milankovic, Carrigtwohill (IE); Jack O'Donovan, Clonakilty (IE); Graham Krumpelmann, Stillwater, MN (US); Matthew Hartigan, Limerick (IE)

(73) Assignee: Stryker European Operations Limited, Carrigtwohill, Co Cork (IE)

(21) Appl. No.: 19/491,242

(22) PCT Filed: Jun. 14, 2024
(86) PCT No.: PCT/IB2024/055852

§ 371 Date: Dec. 8, 2025

Related U.S. Application Data
(60) Provisional application No. 63/521,424, filed on Jun. 16, 2023.

(51) Int. Cl. A61B 17/32; A61B 17/00; A61B 34/20

(52) U.S. Cl. CPC A61B 17/320016; A61B 34/20; A61B 2017/00946; A61B 2034/2051

(57) ABSTRACT

A cutting accessory for a surgical cutting instrument. The cutting accessory includes a sensor assembly including at least one sensor coupled near a cutting tip of a tube assembly. The tube assembly may be straight, bent, rigid, or malleable. The sensor assembly may include a substrate coupled to the outer tube with electrical traces disposed thereon in a twisted pair configuration. The sensor may be a coil sensor wound about an insulative spacer coupled to a distal region of the substrate. The coil sensor is electrically coupled to the electrical traces. A flexible region of the substrate may traverse the bend or malleable region, and the electrical traces may be in a linear configuration within the flexible region. The sensitivity of the coil sensor may be based on the properties of the tube assembly, and/or the axial location of the coil sensor relative to the cutting tip.

### PRIORITY CLAIM

This application claims priority to and all the benefits of United States Provisional Patent Application No. 63/521,424, filed on Jun. 16, 2023, the entire contents being hereby incorporated by reference.

### BACKGROUND

Powered surgical cutting instruments are ubiquitous in the modern surgical suite and used to resect nearly all tissue types in nearly all anatomical locations. The form of the cutting instrument may be based in part on the accessibility of the tissue to be resected. For procedures involving difficult-to-access anatomy, such as the ear, nose, and throat (ENT), a shaft of the cutting instrument may include at least one bend or curve. The bend may be rigid at a fixed angle, or the angle may be selectively adjustable through articulation or manual bending of the shaft. The rigid bend often requires selection of the desired cutting implement from a catalogue of cutting tools, and therefore does not easily permit the surgeon to make on-the-fly adjustments during the surgical procedure.

Of particular interest is tracking and navigation of the cutting instrument, and more particularly of the cutting tip of the angled cutting instrument. Accurate tracking is essential to provide precise information to the user on the location of the cutting tip relative to sensitive anatomy, such as orbital cavity, carotid artery, optical nerve, and the like. Current solutions are fraught with shortcomings. For example, United States Patent Publication No. 2010/0234724, published Sep. 16, 2010, hereby incorporated by reference, discloses a clamp including a sensor for electromagnetic (EM) tracking. The clamp is removably couplable on a proximal portion of the shaft adjacent to the hub. The solution requires providing a separate component, and therefore onerous assembly and calibration prior to initiation of the surgical procedure. Further, the size of the clamp diminishes line of sight, and its proximal positioning is generally incompatible with non-rigid shafts. Another example is disclosed in United States Patent Publication No. 2020/0107885, published Apr. 9, 2020, hereby incorporated by reference, in which a position sensor is near the cutting tip of the shaver. However, the distal end of a cutting tip is typically rounded with a cutting window located proximal to the distal end. As a result, the rounded geometry may distort perception of the tracked point being when being displayed on a monitor, which may lead to inaccuracies in tissue resection. Therefore, there is a need in the art to provide for improved, accurate tracking of a feature of the cutting tip on a cutting accessory that is removably couplable to a capital handpiece, and/or with the shaft being selectively adjustable through manual bending. It would be also desirable to provide for rotation of a cutting window on a tube assembly that is articulable or bendable.

### SUMMARY

The present disclosure is directed to a cutting accessory for a surgical cutting instrument. The cutting accessory facilitates navigation in an intuitive and accurate manner. A sensor assembly includes a sensor disposed near the cutting tip, and in certain implementations, distal to a bend and/or a malleable region. Therefore, a position of the sensor, relative to the cutting tip, is fixed regardless of whether the bend is altered or reoriented. In addition, cutting accessories with longer shafts may be tracked more accurately. The sensor assembly is designed to reduce susceptibility to electromagnetic noise, for example, from the motor disposed within the handpiece. The advanced functionality discussed below is integrated on a cutting accessory that is removably couplable to a handpiece. Additional advantages of the cutting accessory will be readily appreciated in view of the written description and accompanying figures.

The cutting accessory includes a hub, and a tube assembly extending distally from the outer hub. The tube assembly includes an outer tube, a drive shaft, and, optionally, an intermediate tube. A cutting tip is at a distal end of the drive shaft. The cutting tip is a sharp or toothed edge rotatably disposed within a cutting window. The drive shaft may be an inner tube defining a suction lumen. Alternatively, a bur head may be coupled to the drive shaft or the inner tube. The tube assembly may be straight, angled, or malleable. The drive shaft includes at least one flexible region configured to transmit torque through a bend or a malleable region.

The cutting accessory includes a sensor assembly with at least one sensor positioned near the cutting tip. The sensor may be a coil sensor. The sensor is positioned distal to the bend or the malleable region such that a position of the sensor is adjacent to or near the cutting tip. The sensor assembly includes a substrate and electrical traces. The substrate is coupled to and extends along the outer tube. The substrate may extend along a lower aspect of the outer tube to traverse a convex side of the bend. The sensor is positioned in a fixed spatial relationship relative to a predetermined point on, or feature of, the cutting tip. The sensor is configured to generate an electrical signal in response to an electromagnetic (EM) field, with the electrical signal indicating a position of the cutting tip in a three-dimensional space.

The coil sensor may be coaxially disposed about the outer tube. The coil sensor may be a five degree of freedom (DOF) sensor wound from wire, such as copper wire. The coil sensor has a sensor axis aligned with an axis of the tube assembly at the cutting tip. In one non-limiting example, the wire is between 48 and 60 American wire gauge (AWG) within the range of approximately 300 to 2,200 windings, and more particularly within the range of approximately 400 to 600 windings. A resulting thickness of the coil sensor is approximately 0.25 millimeters. Further, the number of windings may be designed to impart the desired sensitivity of the sensor assembly, which is affected by the properties of the tube assembly. The sensitivity of the sensor assembly may also be adjusted based in part on the axial position of the coil sensor on the outer tube.

The substrate may be formed from a polymer such as polyimide (PI) or other suitably flexible material. The substrate may be elongate and include a proximal region, a flexible region, and a distal region. The flexible region may be narrower than the proximal region and/or the distal region. The flexible region is axially aligned with the bend or the malleable region of the tube assembly. The proximal region is positioned between the flexible region and the hub of the cutting accessory. The distal region is distal to the flexible region and positioned near or adjacent to the cutting tip.

A pair of the electrical traces extend along a length of the substrate. The electrical traces are arranged in a twisted pair configuration. The twisted pair configuration may be associated with a first electrical trace and a second electrical trace in which vias provide for the traces to alternate between opposing sides of the substrate. Each of the electrical traces are arranged in complementary linear segments on opposing upper and lower surfaces of the substrate with the linear segments being separated by the vias. The pitch of the twists may be between approximately 2.3 and 3.3 millimeters, and more particularly approximately 2.8 millimeters.

The twisted pair configuration may be disposed in or extend along the proximal region of the substrate. A linear configuration may be disposed in or extend along the flexible region of the substrate. The linear configuration may include each of the electrical traces being arranged in a complementary linear pathway on the opposing upper and lower surfaces of the substrate. A second twisted pair configuration may be positioned distal to the linear configuration and near the sensor.

The sensor assembly includes coil pads disposed in the distal region of the substrate and formed from conductive material. The electrical traces are electrically coupled to the coil pads. The sensor is coupled to the coil pads to provide electrical communication between the sensor and the electrical traces. One of the coil pads may be disposed on the upper surface of the substrate, and the other one of the coil pads may be disposed on the lower surface of the substrate. The sensor assembly further includes proximal pads disposed in the proximal region of the substrate and formed from conductive material. The electrical traces are electrically coupled to the proximal pads. Within the hub, a sensor cable may be electrically coupled to the proximal pads. Alternatively, a wireless transmitter may be electrically coupled to the proximal pads and configured to transmit data to a wireless receiver of the navigation system.

The sensor assembly may include an insulative spacer coupled to the substrate and coaxially disposed about the outer tube. The insulative spacer is a tubular segment formed from a polymer such as polyimide. The coil sensor is wound about the insulative spacer. The substrate is coupled to the insulative spacer. The insulative spacer is coaxially disposed about the outer tube, and the coil sensor is coaxially disposed about the insulative spacer. An insulative layer or sheath may be coaxially disposed about the coil sensor, the substrate, and the electrical traces. The sheath may extend proximally from over the coil sensor to a position near or within the hub. The sheath may be a heat-shrink material, a polymeric jacket secured to the outer tube, or the like.

The cutting accessory provides for ergonomic handling and manipulation, including rotation of the cutting window. The rotation of the cutting window may be implemented through an actuator operably coupled to the tube assembly. For improved ergonomics, the actuator may be a dial operably coupled to a crown or upper aspect of the outer hub and distally oriented at an acute angle relative to a longitudinal axis of the tube assembly. Other variants are disclosed herein.

Therefore, according to a first aspect of the present disclosure, the cutting accessory includes the hub configured to be removably coupled to the handpiece of the powered surgical instrument. The outer tube extends from the hub, and the drive shaft is coaxially and rotatably disposed within the outer tube. The cutting tip is disposed at the distal end of the drive shaft. The sensor assembly is configured to be arranged in electronic communication with the navigation system. The sensor assembly includes the substrate coupled to and extending along the outer tube. The substrate comprises the flexible region, and the proximal region between the flexible region and the hub. The electrical traces extend along the substrate. The electrical traces are arranged in the twisted pair configuration in the proximal region, and the linear configuration in the flexible region. The coil sensor is coaxially disposed about the outer tube between the flexible region and the cutting tip. The coil sensor is in electrical communication with the electrical traces. The coil sensor is configured to detect changes in an electric field induced by the navigation system.

According to a second aspect of the present disclosure, the cutting accessory includes the hub configured to be removably coupled to the handpiece of the powered surgical instrument. The outer tube extends from the hub. The drive shaft is coaxially and rotatably disposed within the outer tube. The cutting tip is disposed at the distal end of the drive shaft. The sensor assembly is configured to be arranged in electronic communication with the navigation system. The sensor assembly includes the substrate coupled to and extending along the outer tube, and the electrical traces extending along the substrate. The coil sensor includes a conductive wire coaxially wound about the outer tube and spaced proximal from a feature of the cutting tip by a calibrated distance. The coil sensor is in electrical communication with the electrical traces. The coil sensor is configured to detect changes in an electric field induced by the navigation system. The sheath may be coaxially disposed about the substrate and the coil sensor.

According to a third aspect of the present disclosure, the cutting accessory includes the hub configured to be removably coupled to a handpiece of the powered surgical instrument. The outer tube extends from the hub, and the drive shaft is coaxially and rotatably disposed within the outer tube. The cutting tip is disposed at the distal end of the drive shaft. The sensor assembly is configured to be arranged in electronic communication with the navigation system. The sensor assembly includes the substrate coupled to and extending along the outer tube, and the electrical traces extending along the substrate. The insulative spacer is coupled to the substrate and coaxially disposed about the outer tube. The coil sensor is coaxially disposed about the insulative spacer. The coil sensor is in electrical communication with the electrical traces. The coil sensor is configured to detect changes in an electric field induced by the navigation system.

According to a fourth aspect of the present disclosure, the cutting accessory includes the hub configured to be removably coupled to a handpiece of the powered surgical instrument. The outer tube extends from the hub and includes a bend or a bendable region. The drive shaft is coaxially and rotatably disposed within the outer tube. The drive shaft is configured to transmit torque through the bend or the bendable region. The cutting tip disposed at a distal end of the drive shaft. The sensor assembly is configured to be arranged in electronic communication with the navigation system. The sensor assembly includes the substrate coupled to and extending along the outer tube. The substrate comprises a proximal region and a flexible region. The width of the flexible region is less than the width of the proximal region, and at least a portion of the flexible region of the substrate is aligned with the bend or the bendable region of the outer tube. The electrical traces extend along the substrate. The coil sensor is coaxially disposed about the outer tube between the flexible region and the cutting tip. The coil sensor is in electrical communication with the electrical traces. The coil sensor is configured to detect changes in an electric field induced by the navigation system.

According to a fifth aspect of the present disclosure, the cutting accessory includes the hub configured to be removably coupled to a handpiece of the powered surgical instrument. The outer tube extends from the hub, and the drive shaft is coaxially and rotatably disposed within the outer tube. The cutting tip is disposed at a distal end of the drive shaft. The sensor assembly is configured to be arranged in electronic communication with the navigation system. The sensor assembly includes a coil sensor coaxially disposed about the outer tube. The coil sensor is formed from a gauge of wire and a number of windings to provide a sensor sensitivity that compensates for the material properties of the outer tube and the drive shaft.

According to a sixth aspect of the present disclosure, a method of assembling the cutting accessory includes providing or cutting the insulative tube to a length corresponding to a designed length of the coil sensor. The conductive wire is wound about the insulative tube to form the coil sensor. The substrate is provided that includes the electrical traces coupled to the substrate. The insulative tube is secured to the substrate. Electrical communication is established between the coil sensor and the electrical traces of the substrate. The insulative tube and the coil sensor are coaxially directed to be disposed over an outer tube of the cutting accessory. The substrate is secured to the outer tube. A sheath may be heat shrunk coaxially about the substrate and the coil sensor.

### BRIEF DESCRIPTION OF THE DRAWINGS

FIG. 1 is a perspective view of a powered surgical cutting instrument. An implementation of a cutting accessory is removably coupled to a handpiece. The cutting accessory includes an outer hub, a tube assembly extending from the outer hub, and a sensor assembly coupled to the tube assembly.

FIG. 2 is a perspective view of the cutting accessory of FIG. 1.

FIG. 3 is a bottom perspective view of the cutting accessory of FIG. 1.

FIG. 4 is an exploded view of the cutting accessory of FIG. 1.

FIG. 5 is a sectional elevation view of the cutting accessory of FIG. 2 taken along lines 5-5.

FIG. 6 is a detailed view of a tip of the cutting accessory within circle 6-6 of FIG. 5.

FIG. 7 is a plan view of a substrate of the sensor assembly.

FIG. 8 is a detailed view of a portion of the substrate of the sensor assembly within circle 8-8 of FIG. 7 to depict electrical traces in twisted pair configuration. Detail A and B of FIG. 8 each show one of the electrical traces respectively.

FIG. 9A is a schematic representation of a first variant of the arrangement of the sensor assembly on the tube assembly.

FIG. 9B is a schematic representation of a second variant of the arrangement of the sensor assembly on the tube assembly.

FIG. 10 shows a third variant of the arrangement of the sensor assembly on the tube assembly in which the twisted pair configuration is positioned distal to a linear configuration of the electrical traces.

FIG. 11 shows a method of assembly of the implementations of the cutting accessory depicted and described herein.

FIG. 12 is a perspective view of a powered surgical cutting instrument. An implementation of a cutting accessory is removably coupled to a handpiece. The cutting accessory includes an outer hub and a tube assembly extending from the outer hub.

FIG. 13 is a rear perspective view of the cutting accessory.

FIG. 14A is a detailed view of the tube assembly of FIG. 13 within circle 14A as modified to depict a first variant of the sensor.

FIG. 14B is a detailed view of a variant of the tube assembly of FIG. 13 within circle 14B as modified to depict a second variant of the sensor.

FIG. 15 is a sectional elevation view of the cutting accessory of FIG. 13 taken along section lines 15-15.

FIG. 16 is an exploded perspective view of the cutting accessory including the outer hub, the tube assembly, and an electrical bridge.

FIG. 17 is a front perspective view of an implementation of the electrical bridge.

FIG. 18 is a rear perspective view of the electrical bridge of FIG. 17.

FIG. 19 is a perspective view of another implementation of the electrical bridge.

FIG. 20 is a perspective view of a powered surgical cutting instrument with another implementation of the cutting accessory removably coupled to a handpiece. A sensor cable extends from the cutting accessory.

FIG. 21 is a sectional elevation view of another implementation of the cutting accessory.

FIG. 22 is a perspective view of another implementation of the electrical bridge.

FIG. 23 is a representation of a navigation system including a processor, a field generator, and a display. The cutting instrument is used in conjunction with the navigation system and the display to provide real-time navigation guidance.

FIG. 24 is an exemplary output of the real-time navigation guidance.

FIG. 25 are representations of visualization options selectable on the display.

FIG. 26 is a partial perspective view of an implementation of the cutting tip in which a tip geometry included a pointed tip providing a tracked point configured to be calibrated to the navigation sensor.

FIG. 27 is an axial view of the cutting tip of FIG. 26.

FIG. 28 is a sectional view of the cutting tip of FIG. 26 taken along section lines 28-28.

FIG. 29 is a perspective of another implementation of the cutting accessory in which a cutting tip includes a bur head that is disposed adjacent to a hood section.

FIG. 30 is a top plan view of the cutting tip of FIG. 29.

FIG. 31 is an elevation view of the cutting tip of FIG. 29.

FIG. 32 is a partially exploded view of the cutting accessory of FIG. 29.

FIG. 33 is a detailed view of the sensor assembly of FIG. 32 within circle 33-33.

FIGS. 34A-34D are implementations of an actuator for rotating the cutting window.

### DETAILED DESCRIPTION

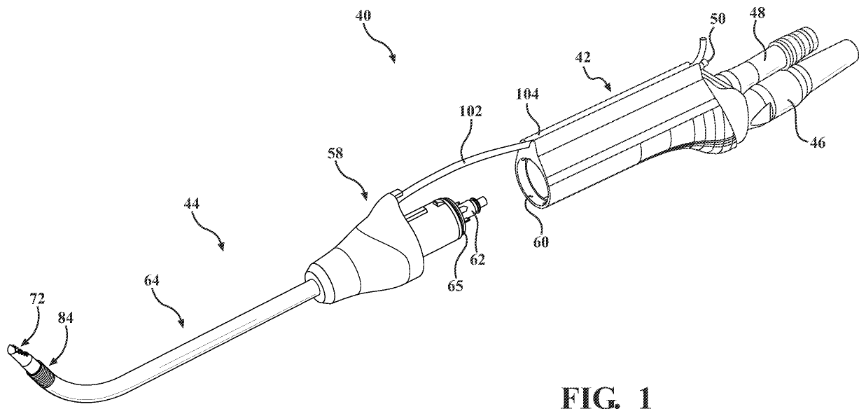

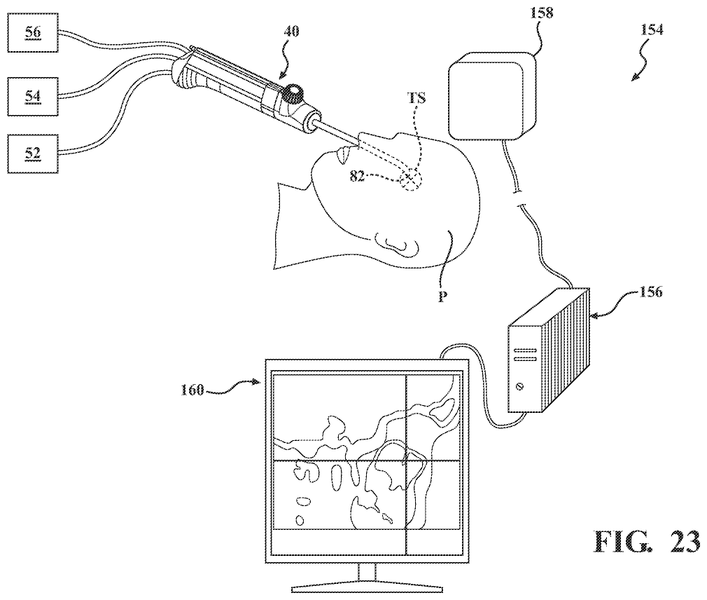

FIG. 1 shows a surgical cutting instrument 40 including a handpiece 42, and a cutting accessory 44 configured to be removably coupled to the handpiece 42. The handpiece 42 is a capital component; i.e., a component configured to be sterilized and reused over many surgical procedures. The cutting accessory 44 may be disposed of after a single use, or it may be manufactured to be sterilizable and reusable as well. Alternatively, the handpiece 42 and the cutting accessory 44 may be integrally formed and not detachable from one another. The handpiece 42 is contoured for ergonomic grasping and manipulation. The handpiece 42 includes a power port or power cord 46, a suction port 48, and, optionally, an irrigation port 50. A motor (not shown) within the handpiece 42 is driven by power transmitted through the power cord 46, which is configured to be removably coupled to a power source 52 (schematically shown in FIG. 23). The power source 52 may be a surgical console, for example, a powered instrument driver sold under the tradename CORE by Stryker Corporation (Kalamazoo, Mich.). The suction port 48 is configured to removably receive a suction tube to establish a suction path with a suction source 54 (schematically shown in FIG. 23) with which the suction tube is configured to be removably coupled. One suitable suction source is disposed on a waste management system sold under the tradename Neptune by Stryker Corporation. In certain implementations, the irrigation port 50 is configured to removably receive an irrigation tube to establish an irrigation path with a source of irrigation liquid 56 (schematically shown in FIG. 23). An irrigation pump (not shown) is configured to direct the irrigation liquid through the handpiece 42 and through the cutting accessory 44 to be discharged at the surgical site. The suction source 54 and/or the irrigation pump may be integrated on the surgical console. The suction and irrigation paths of the surgical cutting instrument 40 may be at least similar to those disclosed in commonly owned International Publication No. WO 2021/224862, published Nov. 11, 2021, and commonly-owned International Publication No. WO 2022/123535, published Jun. 16, 2022, the entire contents of each being hereby incorporated by reference.

The cutting accessory 44 includes a hub, also referred to herein as an outer hub 58. The handpiece 42 and the outer hub 58 of the cutting accessory 44 include complementary coupling features (not identified) to releasably secure the cutting accessory 44 to the handpiece 42. The coupling features may be a latch or other suitable interlocking geometries, for example, those disclosed in the aforementioned International Publication No. WO2021/224862. The handpiece 42 defines at least one opening or cavity 60, and the cutting accessory 44 may include a drive hub 62 configured to be directed to within the cavity 60. A drive hub 62 is rotatably disposed within the outer hub 58 and includes at least one spline or interfacing geometry configured to be operably coupled with the motor, and with the outer hub 58 to be coupled to the handpiece 42. A seal 65 may be coupled to or disposed within the outer hub 58 to create a fluid-tight connection between the outer hub 58 and the handpiece 42 for delivery of the irrigation fluid through the cavity 60 and into an annular space defined between the drive hub 62 and the outer hub 58.

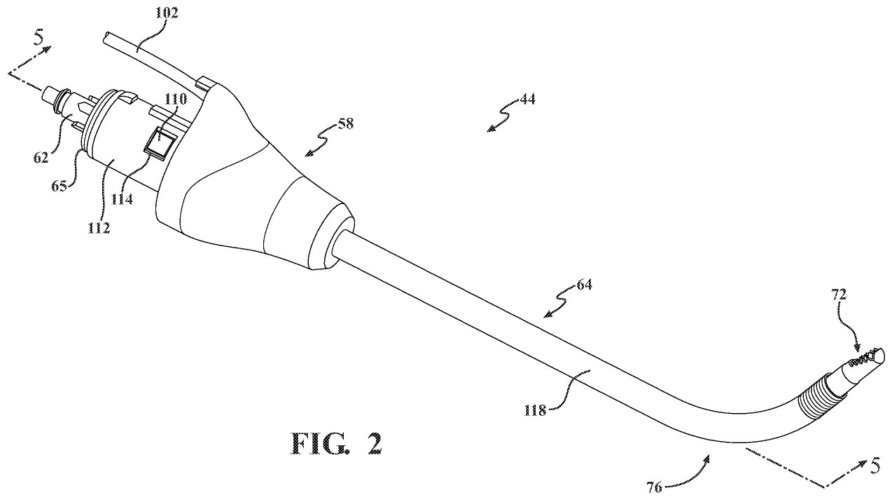

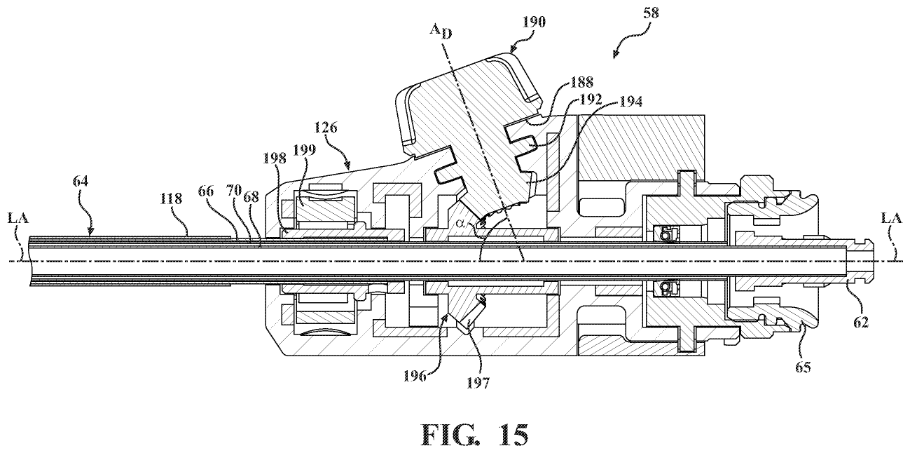

With further reference to FIGS. 2-6, a tube assembly 64 extends distally from the outer hub 58 and includes an outer tube 66, a drive shaft 68 (also referred to herein as an inner tube), and, optionally, an intermediate tube 70 (see FIG. 15). The outer tube 66 is coupled to the outer hub 58, the intermediate tube 70 is rotatably and coaxially disposed within the outer tube 66, and the drive shaft 68 is rotatably and coaxially disposed within the intermediate tube 70 and coupled to the drive hub 62. A cutting tip 72 is at a distal end of the drive shaft 68. In implementations in which the cutting accessory 44 is a microdebrider/shaver, the drive shaft 68 is an inner tube defining a suction lumen, and the cutting tip 72 is a sharp or toothed edge disposed on the inner tube. The outer tube 66 or the intermediate tube 70 may define the cutting window 74 with the inner tube rotatable therein. As a result, with the drive hub 62 operably coupled to the motor, the motor rotates the inner tube to cause the toothed edge to shear or debulk the tissue within the cutting window 74. The resected tissue is suctioned through the cutting window 74 and into the suction lumen. In implementations in which the cutting accessory 44 is a bur, the bur head is coupled to the drive shaft 68 or the inner tube and may extend beyond a distal end of the outer tube 66. The bur may be a diamond bur, fluted bur, or any other suitable type and size of bur head. It is also contemplated that aspects of the present disclosure may be used with other cutting implements such as a curette, rasp, blade tip, trephine, brush, or the like, or non-cutting manual or powered instruments such as a screwdriver, endoscopic camera, light assembly, suction instrument, or the like.

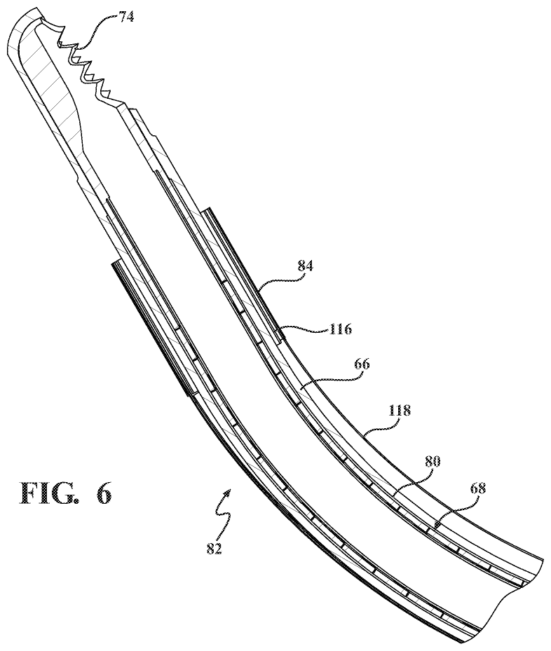

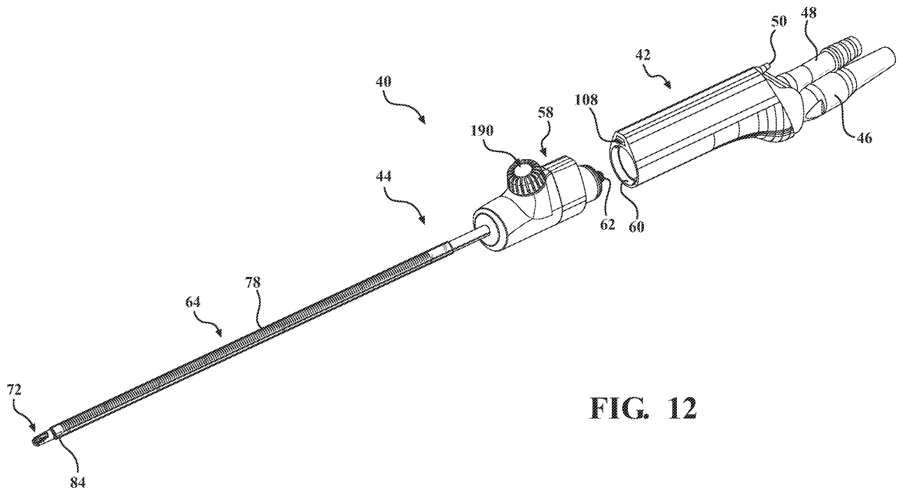

The tube assembly 64 may be straight, angled, or malleable. The angled or malleable variants of the tube assembly 64 may include at least one bend 76 or at least one bendable or malleable region 78 (see FIG. 12). The drive shaft 68 includes at least one flexible region 80 (see FIG. 6) corresponding at least to an axial location of the bend 76 or the malleable region 78. The flexible region 80 of the drive shaft 68 is configured to conform to the shaped configuration maintained by the outer tube 66 and transmit torque through the bend 76. The flexible region 80 may be accomplished through several suitable manners, such as those disclosed in the aforementioned International Publication No. WO2022/123535. In one example, the flexible region 80 includes castellated segments interlocked with one another to define slots. The interlocking of the segments is configured to transmit torque with rotation of the drive shaft 68 by the motor. Additionally, or alternatively, the drive shaft 68 may include helical, spiral, wound, or braided characteristics configured to transmit torque about the bend(s) 76 of the tube assembly 64 in the shaped configuration. It is further contemplated that the tube assembly 64 may be modified to be articulable through suitable means, for example, those disclosed in commonly owned United States Patent Publication No. 2018/0242962, published Aug. 30, 2018, the entire contents of which are hereby incorporated by reference.

In a two-tube arrangement in which suction and irrigation is provided, a first liner (not shown) may be coupled to the outer tube 66 and disposed over the slots to prevent egress of the irrigation fluid through the slots of the outer tube 66. The first liner may be a heat-shrink tubing disposed over an outer surface of the outer tube 66, or a tubular jacket coupled to the outer surface or an inner surface of the outer tube 66. Likewise, a second liner (not shown) may be coupled to and disposed over or within the flexible region(s) 80 of the drive shaft 68. In implementations in which there is an intermediate tube 70, the intermediate tube 70 also includes at least one flexible region (not identified) corresponding at least to the axial location of the bend(s) 76 or the malleable region(s) 78, and the axial location of the flexible region(s) 80 of the drive shaft 68. A third liner (not shown) may be provided, coupled to, and disposed over or within the flexible region(s) of the intermediate tube 70.

The navigation of surgical instruments is becoming increasingly commonplace in the modern surgical suite. Known devices enabling navigation often require a relatively large sensor unit to be positioned on or near the handpiece and external to the anatomy within which the cutting tip is disposed. Such solutions result in suboptimal accuracy, particularly in cutting instruments with a longer tube assembly and/or a bend along the length of the tube assembly. Moreover, such solutions are incompatible with the on-the-fly adjustment afforded by a malleable surgical instrument. In other words, the known devices undesirably require recalibration of the navigation software after each instance the tube assembly is bent or re-bent.

The cutting accessory 44 of the present disclosure overcomes such shortcomings by including a sensor assembly 82 with at least one sensor 84 positioned near the cutting tip 72. More particularly, the sensor 84 is positioned distal to the bend 76 or the malleable region 78 such that a position of the sensor 84 relative the cutting tip 72 is static. Further, with the sensor 84 being adjacent to or near the cutting tip 72, superior accuracy is possible. Still further, the construction of the sensor assembly 82 is minimally sized to preserve line of sight of the cutting tip 72 (e.g., in endoscopic procedures) and otherwise provide a navigation-enabled instrument with an appearance and workflow akin to surgical instruments familiar to surgeons.

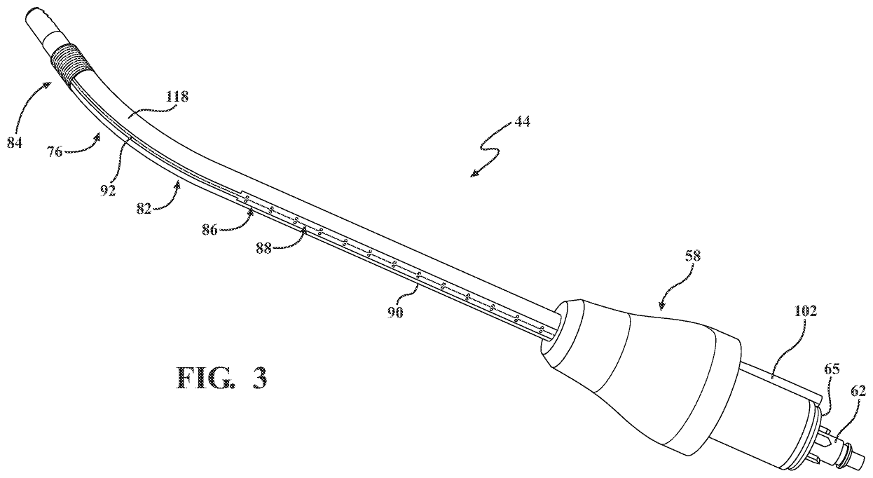

FIGS. 1-6 show the sensor 84 being a coil sensor. The sensor assembly 82 further includes a substrate 86, and electrical traces 88. The substrate 86 is coupled to and extends along the outer tube 66. For example, FIG. 3 shows the substrate 86 being strip-like in form and extending along a lower aspect of the outer tube 66 to traverse a convex side of the bend 76. In alternative implementations in which the tube assembly 64 is malleable, the substrate 86 may be positioned to extend along a malleable spine (i.e., between opposing slotted portions) of the malleable region. The sensor 84 is coupled to the substrate 86 at a position distal to the bend 76. In manners to be described, the sensor 84 is positioned in a fixed spatial relationship relative to a predetermined point on or feature of the cutting tip 72, for example, a distalmost point. The sensor 84 is configured to generate an electrical signal in response to an electromagnetic (EM) field with the electrical signal indicating a position of the cutting tip 72 in a three-dimensional space. In other words, the sensor 84 is configured to detect changes in an electric field induced by a field generator 158 of a navigation system 154 (see FIG. 23).

A preferred implementation of the sensor is a coil sensor 84 coaxially disposed about the outer tube 66. Alternatively it is contemplated that the coil sensor 84 may be coaxially disposed within or between the drive shaft 68 and the outer tube 66 of the tube assembly 64. The coil sensor 84 may be a five degree of freedom (DOF) sensor wound from wire, such as copper wire, optionally coated in a suitable coating such as Parlene. As a result, the coil sensor 84 has a sensor axis aligned with an axis of the tube assembly 64 at the cutting tip 72. In one non-limiting example, the wire is between 48 and 53 American wire gauge (AWG) within the range of approximately 300 to 2,200 windings, and more particularly within the range of approximately 400 to 600 windings. The arrangement results in a net increase in diameter of the tube assembly 64 as little as 0.5 millimeters. Again, this minimizes the impact on the line of sight of the surgeon while permitting the coil sensor 84 to be disposed near the cutting tip 72 for superior accuracy. The number of windings may be designed to impart the desired sensitivity of the sensor assembly 82, which is affected by the properties (i.e., size, material, etc.) of the tube assembly 64. In one example, the sensitivity of the sensor assembly 82 is within the range of approximately 0.060 to 0.150 V/Hz/T, and more particularly within the range of approximately 0.071 to 0.106 V/Hz/T, which is reduced by approximately 35% to compensate for the properties of the tube assembly 64. Moreover, the sensitivity of the sensor assembly 82 may also be adjusted based in part on the axial position of the coil sensor 84 on the outer tube 66. For example, the sensitivity may be reduced by a greater amount the nearer the coil sensor 84 is to the cutting tip 72.

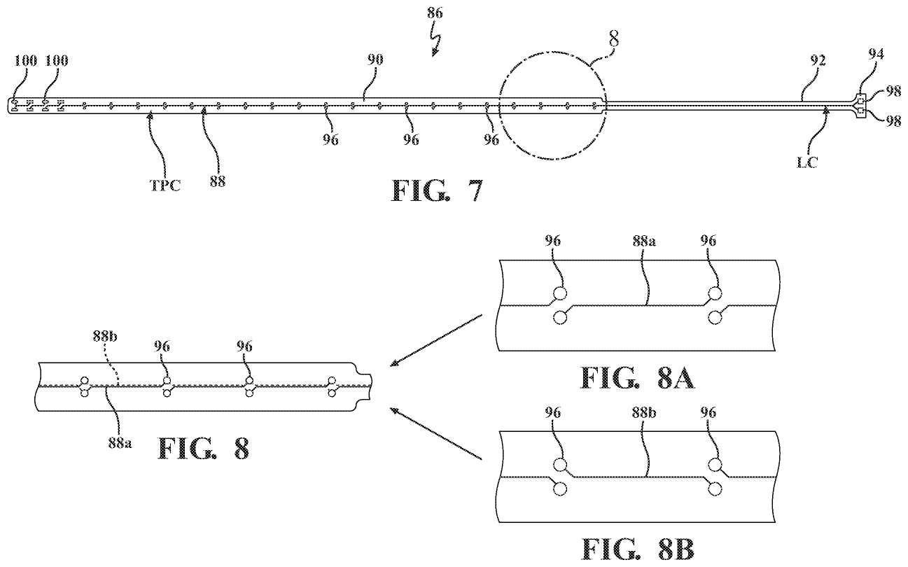

The substrate 86 may be formed from a polymer such as polyimide (PI) or another suitably flexible material. With further reference to FIG. 7, the substrate 86 is elongate and may include a proximal region 90, a flexible region 92, and a distal region 94. The flexible region 92 may be narrower than one or both of the proximal region 90 and the distal region 94. In one example, the width of the flexible region 92 is no greater than two millimeters, the width of the proximal region 90 is no greater than three millimeters, and/or the width of the distal region 94 is no greater than four millimeters. These dimensions are merely exemplary and may be sized to correspond to the dimensions of the tube assembly 64. The flexible region 92 is axially aligned with the bend 76 or the malleable region 78 of the tube assembly 64. The narrower width of the flexible region 92 is to permit sufficient curvature when bending as to prevent kinking during the coupling of the sensor assembly 82 to the bend 76, and/or during repeated bending of the malleable region 78. The proximal region 90 of the substrate 86 is proximal to the flexible region 92, and more particularly positioned between the flexible region 92 and the outer hub 58 of the cutting accessory 44. The distal region 94 of the substrate 86 is distal to the flexible region 92, and more particularly is positioned near or adjacent to the cutting tip 72.

A pair of the electrical traces 88 extend along a length of the substrate 86. With continued reference to FIG. 7 and further reference to FIGS. 8A and 8B, the electrical traces 88 are arranged in a twisted pair configuration (TWP). The twisted pair configuration may be associated with a first electrical trace 88a and a second electrical trace 88b in which vias 96 provide for the traces 88a, 88b to alternate between opposing sides of the substrate 86. In other words, each of the electrical traces 88a, 88b are arranged in complementary linear segments on opposing upper and lower surfaces of the substrate 86 with the linear segments being separated by the vias 96, wherein each of the electrical traces 88a, 88b is routed between the upper and lower surfaces. A suitable construction of the twisted pair configuration is also disclosed in U.S. Pat. No. 5,646,368 issued Jul. 8, 1997, the entire contents of which are hereby incorporated by reference. Among other advantages, routing the electrical traces 88 in the twisted pair configuration minimizes susceptibility to external electromagnetic noise, further maximizes coupling between the electrical traces 88 and prevents emission. The pitch of the twists—i.e., a spacing between the vias 96—may be designed to provide a maximum number of twists for the length of the substrate 86 in view of added costs and potential for rupture of the vias 96 under mechanical stress and strain. In an exemplary implementation, the spacing between the vias 96 is between approximately 2.3 and 3.3 millimeters, and more particularly approximately 2.8 millimeters.

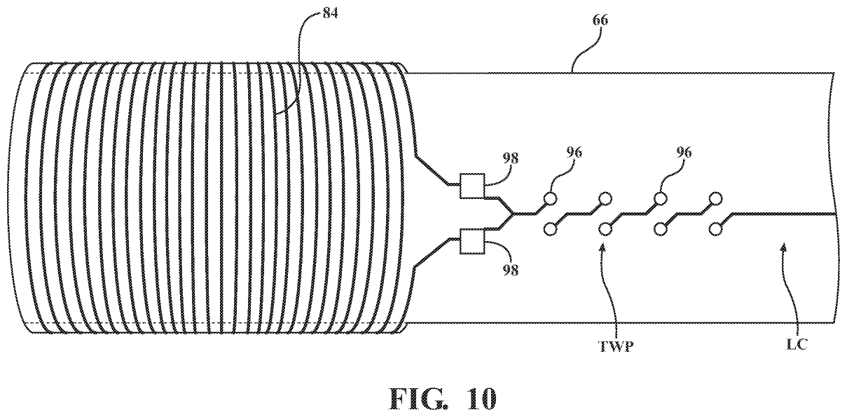

As shown in FIG. 7, the twisted pair configuration may be disposed in or extend along the proximal region 90 of the substrate 86. A linear configuration (LC) may be disposed in or extend along the flexible region 92 of the substrate 86. As mentioned, the vias 96 are typically not suitable for bending. Therefore the linear configuration advantageously accommodates such bending while still providing for the twisted pair configuration along a majority of the length of the sensor assembly 82. The linear configuration may include each of the electrical traces 88a, 88b being arranged in a complementary linear pathway on the opposing upper and lower surfaces of the substrate 86. Another configuration is depicted in FIG. 10 in which a second twisted pair configuration may be positioned distal to the linear configuration and near the sensor 84.

The sensor assembly 82 includes distal pads, also referred to herein as coil pads 98. The coil pads 98 are disposed in the distal region 94 of the substrate 86 and formed from conductive material. The electrical traces 88 are electrically coupled to the coil pads 98, for example, through soldering or another suitable joining means. In particular, one of the electrical traces 88a is coupled to one of the coil pads 98, and another one of the electrical traces 88b is coupled to the other one of the coil pads 98, as best shown in FIG. 10. Further, the sensor 84 is coupled to the coil pads 98 to provide electrical communication between the sensor 84 and the electrical traces 88. An end of the sensor 84 may be coupled to one of the coil pads 98, and another end of the sensor 84 may be coupled to the other one of the coil pads 98. One of the coil pads 98 may be disposed on the upper surface of the substrate 86, whereas the other one of the coil pads 98 may be disposed on the lower surface of the substrate 86.

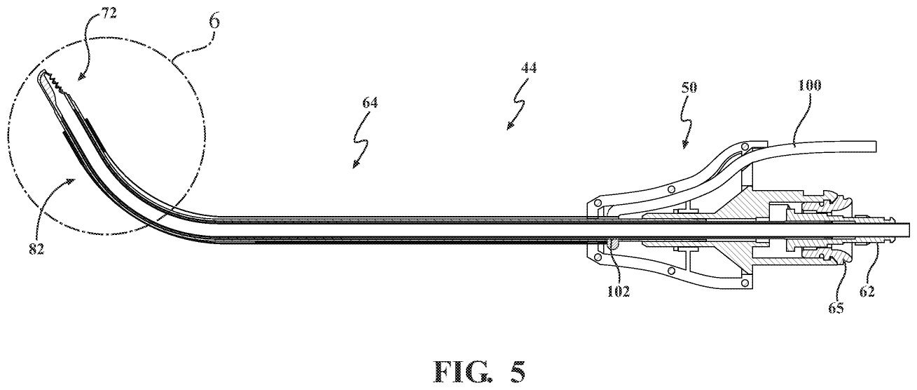

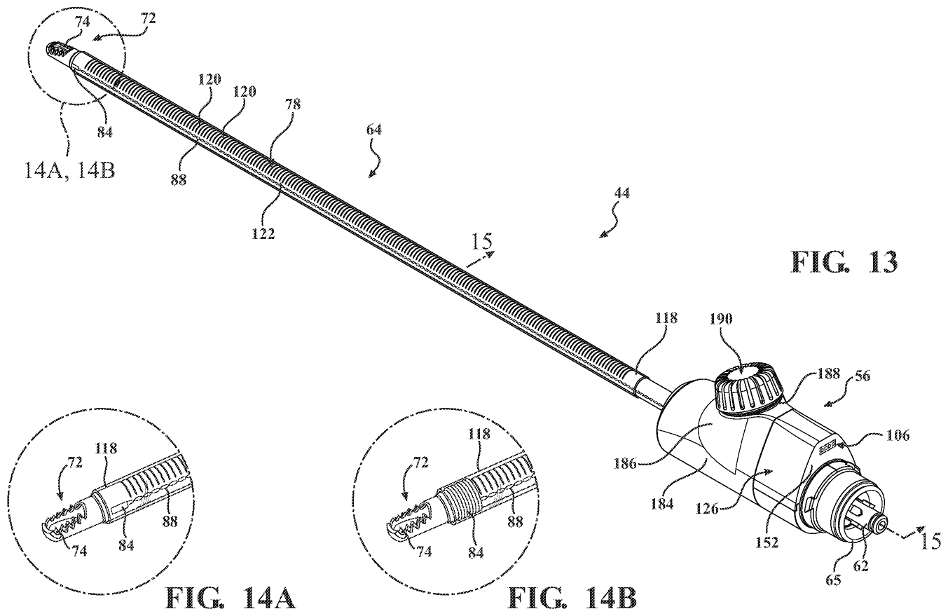

The sensor assembly 82 further includes proximal pads 100 disposed in the proximal region 90 of the substrate 86, and formed from conductive material. The electrical traces 88 are electrically coupled to the proximal pads 100, for example, through soldering or another suitable joining means. The illustrated implementation in FIG. 7 shows four pairs of the proximal pads 100 with each of the electrical traces 88a, 88b coupled to a respective one of the four pairs of the proximal pads 100. Within the outer hub 58, a sensor cable 102 may be electrically coupled to the proximal pads 100, as best represented in the sectional view of FIG. 5. Again, the electrical coupling may be through soldering or another suitable joining means. The sensor cable 102 may include one, two, three, four, or more wires with each wire being electrically coupled to one of the four proximal pads 100. In one example, two wires of the sensor cable 102 are for transmitting sensor signals, and another two wires of the sensor cable 102 are for communication from memory of calibration data, authentication data, and/or identification data. The sensor cable 102 may be routed through a slot 104 of the handpiece 42 (see FIG. 1) and include a plug (not shown) configured to be removably coupled to complementary hardware of the navigation system 154. Alternatively, an electrical connector 106 (see FIG. 16) may be in electrical communication with the sensor assembly 82 and configured to be removably coupled to a complementary electrical connector 108 on the handpiece 42 (see FIGS. 12 and 13). In another variant, a wireless transmitter (not shown) may be electrically coupled to the proximal pads 100 and configured to transmit data to a wireless receiver of the navigation system 154, thereby obviating the need for the sensor cable 102. In certain implementations, the calibration data, authentication data, and/or identification data may be facilitated by a radiofrequency identification (RFID) tag 110 coupled to the cutting accessory 44. FIG. 2 shows that the outer hub 58 may include a proximal housing 112 configured to be inserted into the cavity 60 of the handpiece 42. The proximal housing 112 may define a recess 114, and the RFID tag 116 may be fixedly supported in the recess 114. The RFID tag 116 may be positioned in an axial and/or radial position to correspond to an antenna (not identified) disposed on or within the handpiece 42.

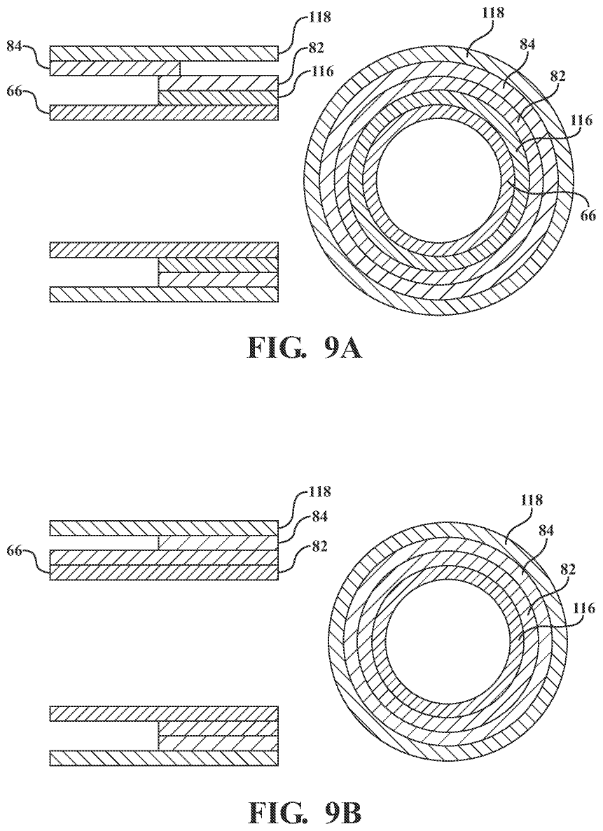

Returning to the coil sensor 84, it is desirable to maintain electrical insulation between the sensor 84 and the outer tube 66, which is typically formed from a conductive material. Therefore, in certain implementations, the cutting accessory 44 may include an insulative spacer 116 coupled to the substrate 86 and coaxially disposed about the outer tube 66. In one exemplary implementation, the insulative spacer 116 is a tubular segment formed from a polymer such as polyimide. The insulative spacer 116 may have a dielectric constant of between 3.3 and 3.5, or other suitable value based on the material properties of the tube assembly 64. During assembly, the coil sensor 84 is wound about the insulative spacer 116. The substrate 86, and more particularly the distal region 94 of the substrate 86, is coupled to the insulative spacer 116. The joining may be facilitated by adhesive or other suitable joining means. The stack up is generally represented in FIG. 9A in which the insulative spacer 116 is coaxially disposed about the outer tube 66, and the coil sensor 84 is coaxially disposed about the insulative spacer 116. Another implementation of the stack up is represented in FIG. 9B in which the substrate 86 includes a distal ring forming a mandrel over which the coil sensor 84 is coaxially disposed.

The cutting accessory 44 may include an insulative layer or sheath 118 coaxially disposed about the coil sensor 84, the substrate 86, and the electrical traces 88. As appreciated from FIGS. 1-6, the sheath 118 may extend proximally from over the coil sensor 84 to a position near or within the hub 58. The sheath 118 may overlay at least a portion of the outer tube 66 and secure the coil sensor 84 and the electrical traces 88 to the outer tube 66. The sheath 118 may be a heat-shrink material, a polymeric jacket secured the outer tube 66, or the like, so as to fix the position of the coil sensor 84 and the substrate 86 in a low-profile manner that does not obstruct visualization of the cutting tip 72 when viewed along the tube assembly 64. The sheath 118 may extend from the outer hub 58, or include a proximal end positioned distal to the outer hub 58 such that a portion of the tube assembly 64 is exposed. In one variant in which the tube assembly 64 is straight and rigid, a rigid auxiliary tube (e.g., a hypotube) may extend from the outer hub 58 and be coaxially disposed over the outer tube 66. The auxiliary tube may be bonded to the outer tube 66, for example, by spot welding, soldering, adhesive, or another suitable joining means.

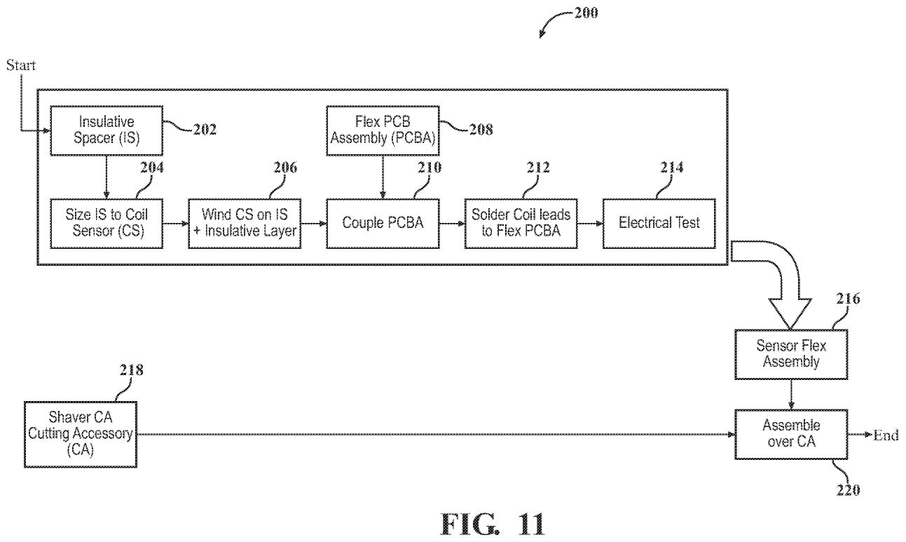

Referring now to FIG. 11, an exemplary method 200 of assembling the cutting accessory 44 is shown. An insulative tube is provided (step 202). The insulative tube may be cut to length to form the insulative spacer 116 sized to a designed length of the coil sensor 84 (step 204). The insulative spacer 116 may have a thickness of approximately 25 micrometers (μm). Conductive wire is wound about the insulative spacer 116 to form the coil sensor 84 (step 206). As mentioned, the wire may be wound between approximately 300 to 2,200 windings, and more particularly within the range of approximately 400 to 600 windings.

A flexible printed circuit board assembly (PCBA) is provided (step 208), which includes the electrical traces 88 coupled to the substrate 86 in the aforementioned twisted pair and linear configurations. The insulative spacer 116 is secured to the flex PCBA (step 210), for example, through an adhesive or the like. In particular, the insulative spacer 116 may be glued to the distal region 94 of the substrate 86. The coil sensor 84, coaxially disposed about the insulative spacer 116, is soldered to the flex PCBA (step 212) to establish electrical communication between the coil sensor 84 and the electrical traces 88 of the substrate 86. For example, ends of the coil sensor 84 may be coupled to the coil pads 98, for example, through soldering. An optional electrical test may be performed (step 214) to ensure the electrical connections are satisfactory. The output may be considered a sensor-flex assembly that is provided for final assembly (step 216). Separately, the cutting accessory 44 may be assembled (step 218), which is provided for final assembly. The sensor-flex assembly is coupled to the cutting accessory (step 220). In particular, the insulative spacer 116 and the coil sensor 84 are coaxially directed to be disposed over the outer tube 66 of the cutting accessory 44. Optionally, the outer tube 66 may include indicia (e.g., laser etching or other marking) to facilitate locating the sensor-flex assembly at the desired location on the outer tube 66 in an intuitive and reproducible manner.

In certain variants, the flexible region 92 of the substrate 86 is positioned axially with the bend 76 or the malleable region 78 of the outer tube 66. The substrate 86 is secured to the outer tube, which may include heat shrinking the sheath 118 coaxially about the substrate 86 and the coil sensor 84. The method may include soldering wires of the sensor cable 102 to the proximal pads 100 to establish electrical communication between the sensor cable 102 and the coil sensor 84.

Referring now to FIGS. 12-14B, another implementation of the cutting accessory 44 is provided in which the tube assembly 64 is configured to be bent and/or re-bent by a user to a shaped configuration. More particularly, at least a portion of the tube assembly 64 may include the malleable region 78 to permit the user to bend it to the shaped configuration, after which the tube assembly 64 is sufficiently sturdy to maintain the shaped configuration despite axial and radial forces associated with deploying the cutting tip 72 at the surgical site. The shaped configuration may range from 1 degree of bend, to 30, 60, 90, or even more degrees of bend. As best shown in FIGS. 12 and 13, the outer tube 66 includes at least one slotted region in which a series of slots 120 form at least one malleable spine 122. The malleable spine 122 is configured to be bent and/or re-bent by a user to a shaped configuration and maintain the tube assembly 64 in the shaped configuration. The cutting accessory 44 provides for the sensor 84 to be coupled to the tube assembly 64 at a position distal to the malleable region 78. As a result, regardless of the nature and quantity of the bends being imparted to the tube assembly 64, data indicative of the location of the cutting tip 72 that is being transmitted from the sensor 84 to the navigation software remains sufficiently accurate, and further does not require recalibration after successive bending events.

The electrical trace(s) 88 extend proximally from the sensor(s) 82 to electronic subcomponents within the outer hub 58. The trace 88 may extend along the malleable spine 122 so as to limit strain with the bending of the tube assembly 64. Additionally, or alternatively, at least a portion of the electrical trace 88 may be arranged in a serpentine configuration (not shown) to further limit strain with bending. The electrical trace 88 may be a twisted wire pair to reduce interference from the sensor 84. As appreciated from FIGS. 12-14A in combination, a second sensor 84 may be coupled on the opposing side of the outer tube 66 with a second electrical trace 88 extending along the opposing malleable spine 122. The sensors 84 may be five DOF sensors oriented at a defined angle between directional vectors of each of the sensors 84. The predefined angle provides information to control rotational position tracking. In another variant, the sensor 84 may be positioned on a lower aspect of the cutting tip 72 opposite to the cutting window 74 so as to avoid impeding the view of the cutting tip 72. Alternatively, FIG. 14B shows the sensor 84 as a coil sensor. Therefore, it should be appreciated that the sensor assembly 82, previously discussed, may be implemented on the tube assembly 64 with the malleable region(s) 78.

Referring to FIGS. 16-21, the sensor assembly 82 may include an electrical bridge 124 and the electrical connector 106. As used herein, the term “bridge” is intended to indicate subcomponents providing electrical communication between the sensor 84 and the electrical connector 106 on a proximal side of the outer hub 58. Unless otherwise indicated, the term is not intended to indicate a bridge circuit. The electrical bridge 124 is disposed within a housing 126 of the hub 58. The electrical bridge 124 includes a board 128, and a ribbon 130 coupled to and extending from the board 128. The electrical connector 106 is coupled to the ribbon 130. The internal geometries of the housing 126 are formed to provide a desired pathway for the board 128 and the ribbon 130 to extend therethrough without interfering with other subcomponents of the cutting accessory 44 to be described.

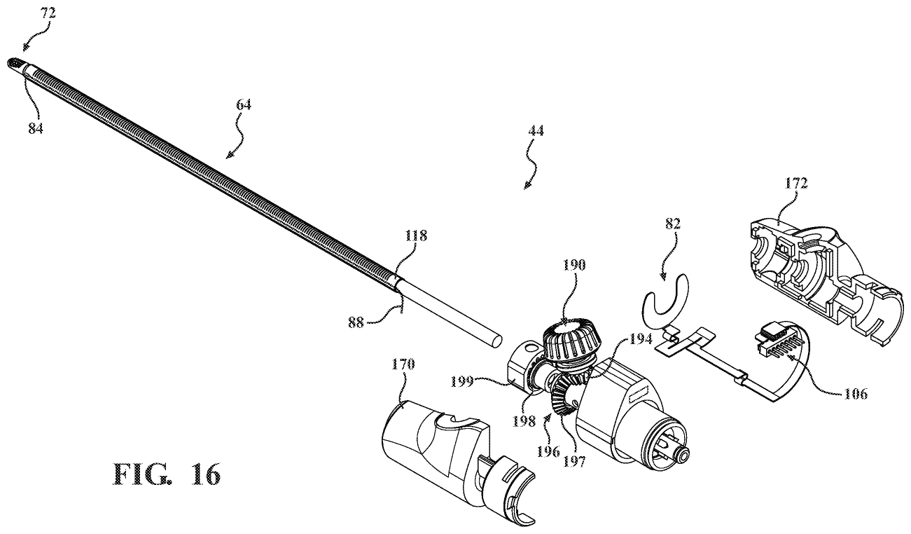

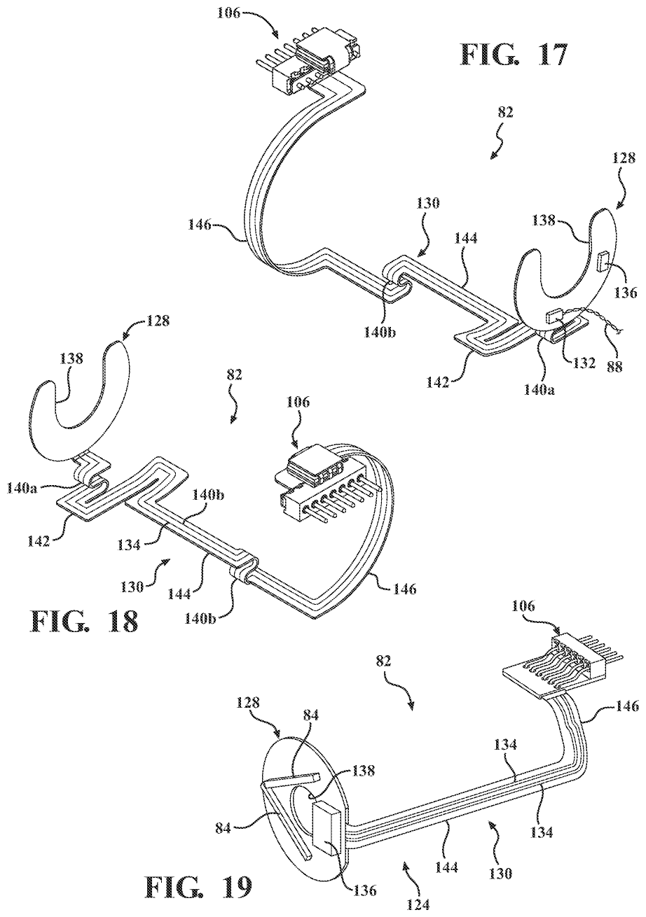

A first implementation of the electrical bridge 124 is illustrated in FIG. 17 and FIG. 18 in which electrical communication is established between the electrical trace 88 and the electrical connector 106. The trace 88 extends proximally along the tube assembly 64 to within the housing 126, and a proximal end of the trace 88 is coupled to the board 128 of the electrical bridge 124, for example, with a pad 132 or other suitable means of fixation. At least two electrical traces 134 extend through the ribbon 130. The electrical connector 106 may be a male pin-type connector with the pins arranged in electrical communication with the traces 134. Alternatively, the electrical connector 106 may be a female pin-type connector with the male pin-type connector disposed on the handpiece 42 (see FIGS. 12 and 13). A six-pin connector may use four pins for the EM signal (two for each five-DOF differential signals), and two pins for communication from memory 136 (one-wire serial communication) of calibration data, authentication data, and/or identification data. The traces 134 may be tightly coupled and routed as a differential pair. In one example, the traces 134 have a width of less than 0.20 millimeters, and wherein a space separation between the tightly coupled traces is less than 0.25 millimeters, thereby reducing susceptibility to external noise.

The electrical bridge 124 forms a flexible circuit configured to accommodate rotation of the outer tube 66, namely in implementations of the cutting accessory 44 in which the orientation of the bend may be altered. The board 128 is fixedly coupled to the outer tube 66, and the ribbon 130 may be formed from a flexible material. The board 128 may define a cutout 138 or opening through which the tube assembly 64 extends. An inner diameter of the cutout 138 may approximate an outer diameter of the outer tube 66. The ribbon 130 may be arranged in a meandering configuration with at least one folded feature 140 configured to furl, unfurl, or otherwise provide slack. The folded feature(s) 140 may be formed by flattened portions of the ribbon 130 doubling back to be stacked on itself. FIGS. 17 and 18 show a first folded feature 140a positioned adjacent and proximal to the board 128. The meandering configuration may include U-shaped sections 142 as shown, and a second folded feature 140b may be disposed within a straightened section 144 extending in the proximal-to-distal direction. A proximal section 146 may extend upwardly in an arcuate manner contoured to an inner surface of the housing 126. The proximal section 146 is coupled to the electrical connector 106.

As the outer tube 66 is rotated within the housing 126, the board 128 rotates in a corresponding manner. Owing to the flexible material forming the ribbon 130 and the nature and arrangement of the first folded feature 140a (and the U-shaped sections 142), the slack provided by the ribbon 130 permits the board 128 to rotate bidirectionally—counterclockwise and clockwise—between predetermined maximum angles associated with bend angle adjustment capabilities of the cutting accessory 44. The predetermined maximum angle may be up to at least 90 degrees.

The sensor assembly 82 may include the memory 136 storing calibration data indicative of the location of the sensor(s) 84 relative to a reference feature of the cutting tip 72, such as an edge, boundary, or a tracked point to be described. In one example, the sensor 84 is positioned proximal to the cutting window 74, as shown in FIGS. 13, 14A and 14B, or positioned distal to the cutting window 74. In implementations in which the tube assembly 64 has the malleable region(s) 78, the sensor 84 is positioned distal to the malleable region 78. It is contemplated that the sensor 84 being positioned near the cutting tip 72 may also be implemented on cutting accessories in which the tube assembly 64 is rigid. The sensor 84 may be at determinable distances along one, two, or three axes relative to the reference feature of the cutting tip 72. In other words, a tracked point to be described may be offset from the sensor 84 along the x-axis, the y-axis, and/or the z-axis. During assembly of the cutting accessory 44, the distance(s) are determined, calibrated, and/or verified, and stored to the memory 136. Therefore, after initializing setup of the navigation system 154, the cutting accessory 44 may be “plug and play” with minimal further input or setup required from the user.

FIG. 19 shows another implementation of the sensor assembly 82 in which two sensors 84 are coupled to the board 128 of the electrical bridge 124. The sensors 84 of the illustrated implementation are two, five DOF sensors oriented at an angle relative to one another. The sensors 84 being disposed within the housing 126 may obviate the need for the trace 88 and the sensor 84 to be near the cutting tip 72. Such an alternative is particularly well suited for implementations in which the tube assembly 64 is rigid (without a bend or with a fixed bend). It is contemplated that the sensors 84 being disposed on the board 128 may be in addition to one or more of the sensors 84 being disposed near the cutting tip 72. It is observed from FIG. 19 that the sensors 84 being coupled to the board 128 obviates the need for the folded features 140 of the ribbon 130, as the position of the sensors 84 relative to the cutting tip 72 is fixed. The board 128 may or may not be fixed to the outer tube 66.

The electrical connector 106 of the cutting accessory 44 is configured to be removably coupled to a complementary electrical connector 108 on the handpiece 42 (see FIGS. 12 and 13). Further, the electrical connector 106 obviates the need for a separate data cable extending from the handpiece 42. The outer hub 58 may include a proximally directed surface 152. The electrical connector 106 is disposed within or extends from the surface 152, and is positioned distal to the drive hub 62 and coupling geometries of the outer hub 58. The axial spacing of the electrical connector 106, relative to the drive hub 62 and the coupling geometries, is such that the electrical connection between the electrical connectors 106, 108 occurs when the mechanical connections are made between the cutting accessory 44 and the handpiece 42. During setup, before or during the procedure, the user need only couple the cutting accessory 44 to the handpiece 42, after which many of the remaining steps may occur automatically. For example, the memory 136 (or other electronic module) may transmit data to a processor 156 of the navigation system 154 (see FIG. 23) for the processor 156 to verify the authenticity of the cutting accessory 44. The data may be transmitted by the RFID tag 110, or alternatively may take the form of a programmed Erasable Programmable Read-Only Memory (EPROM).

If the cutting accessory 44 is a non-genuine article, a prompt may be displayed on a display 160 of the navigation system 154, and/or the processor 156 may prevent the power source 52 from operating the surgical cutting instrument 40. If the authenticity of the cutting accessory 44 is verified, the calibration data may be transmitted from the memory 136 to the processor 156. The calibration data includes the position—i.e., an offset in one, two, or three dimensions—of the tracked point relative to the sensor(s) 84. The calibration data may include other data, such as identification data indicative of the type of cutting accessory 44, which may be used to provide type-specific options and operating parameters on the display 160.

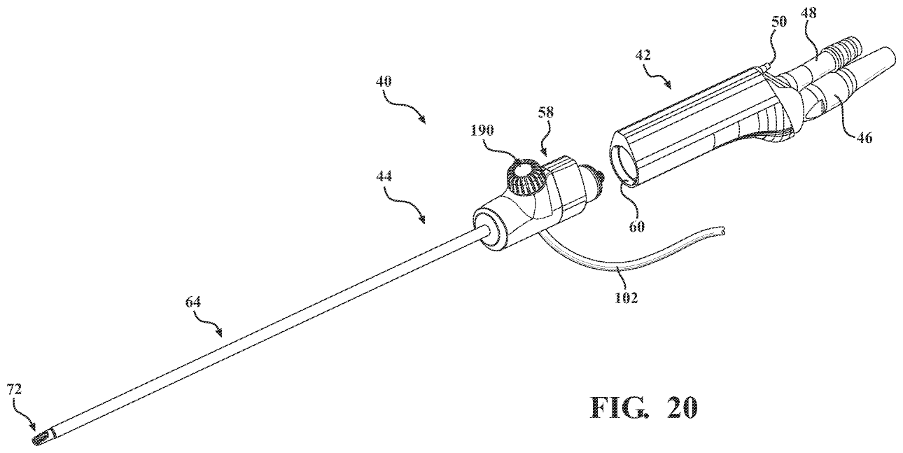

Referring to FIGS. 20-22, another implementation of the hub 58 is shown in which the sensor assembly 82 includes the sensors 84 coupled to the board 128. Unlike the previous implementations, the cutting accessory 44 may not include the electrical bridge 124, nor the electrical connector 106 on the hub 58. Rather, the sensor cable 102 may be coupled to the board 128 and extend through an opening 148 defined by the housing 126. On an opposite end, the sensor cable 102 may include a plug (not shown) configured to be removably coupled to complementary hardware of the navigation system 154. A flange 150 may extend from a lower aspect of the board 128 for supporting a junction between the sensor cable 102 and the board 128. One or more tube management clips (not shown) may also be provided.

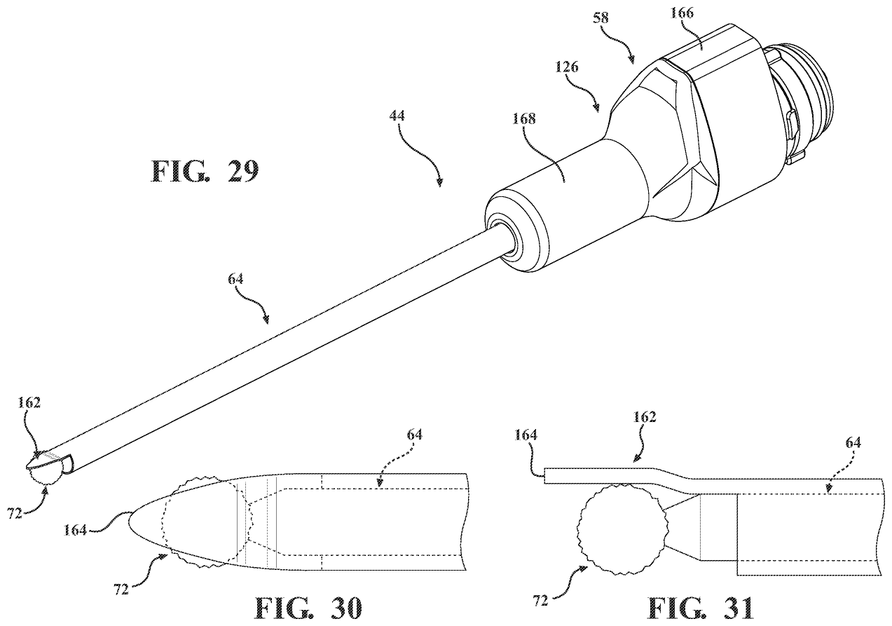

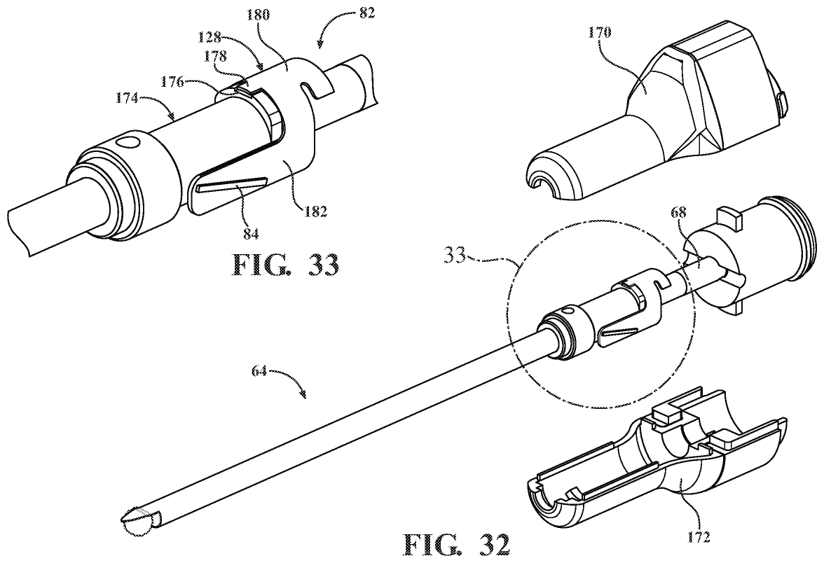

FIG. 29 shows the housing 126 of the cutting accessory 44 including a body 166, and a neck 168 extending from the body 166. With the bur implementation not requiring the gearing and other mechanisms to provide for window rotation, the neck 168 may be relatively narrower than the body 166, and sized to accommodate the fingers of the surgeon for improved grip while burring at high speeds. Owing to the slimmer profile of the neck 168, another implementation of the sensor assembly 82 is shown in FIGS. 32 and 33. FIG. 32 depicts housing sections 170, 172 exploded to show the sensor assembly 82 being coupled to a collar 174 that is secured to the outer tube 66. In particular, the collar 174 includes a proximal flange defining a slot 176 or other anti-rotation feature. The board 128 of the sensor assembly 82 includes a protrusion 178, or another complementary feature, configured to engage the slot 176. The engagement prevents rotation of the board 128 relative to the collar 174, which is rotatably secured to the outer tube 66. The drive shaft 68 is configured to rotate within the outer tube 66, and thus further configured to rotate relative to the collar 174. Therefore, the sensor assembly 82 remains in a fixed position relative to the cutting tip 72. Among other advantages, the sensor assembly 82 of the present implementation is suitably compact to be accommodated in the neck 168 of the housing 126.

The board 128 may be flexibly designed to wrap around components and align with the central longitudinal axis. The board 128 may include a central portion 180, and wing portions 182 (one shown) extending from the central portion 180. The protrusion 178 may be disposed on the central portion 180. The wing portions 182 may extend arcuately about and generally are contoured to opposing sides of the collar 174 or the outer tube 66. Each of the sensors 84 (one shown) may be disposed on a respective one of the wing portions 182. A sensor cable (not shown) may be coupled to the board 128 and extend through an opening defined by the housing 126. The sensor cable may include a plug (not shown) configured to be removably coupled to complementary hardware of the navigation system 154.

The implementations of the cutting accessory 44 are used with a navigation system 154, particularly in an intuitive and plug-and-play manner. Referring now to FIG. 23, a navigation system 154 includes the processor 156, at least one field generator 158, and the display 160. The processor 156 is operable to drive the field generator(s) 158 to generate an EM field of different frequencies around the head of the patient (P). The sensor 84 generates the electrical signal based on the altered EM field. The electrical signal is transmitted from the sensor assembly 82, between the electrical connectors 106, 108, if applicable, and to the processor 156. The processor 156 is configured to determine positional data based on the electrical signals received from the sensor assembly 82 to determine the position of the sensor 84 in three-dimensional space. Further, based on the calibration data, the processor 156 determines the position of the reference feature (e.g., the tracked point) in three-dimensional instrument space.

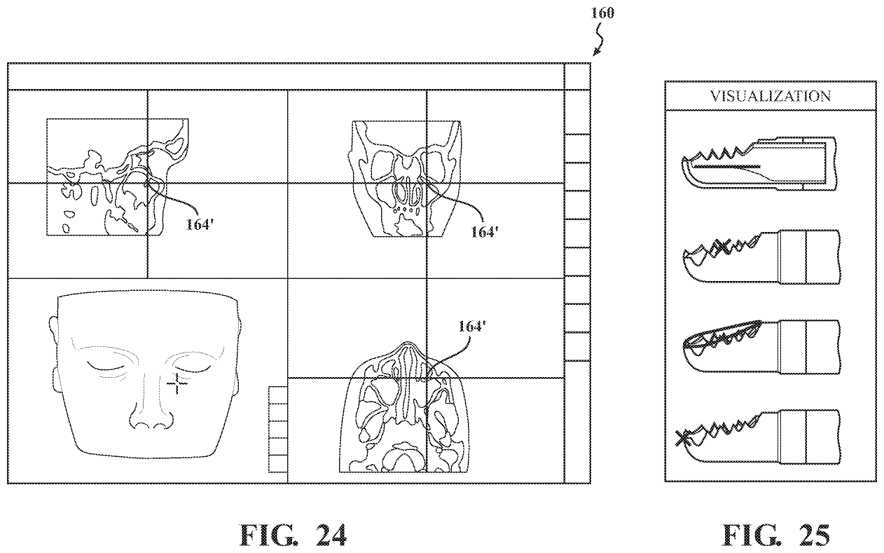

A pre- or perioperative image of the patient anatomy may be received by the processor 156, such as a computed tomography or magnetic resonance imaging scan. Through means known in the art, the scan may be registered to the three-dimensional instrument space. For example, a position of the field generators 158 may be fixed and determined in the three-dimensional instrument space. As a result, the navigation system 154 is configured to display on the display 160 a representation of the cutting instrument 40 relative to the patient anatomy in real time. FIG. 24 shows a representative output of the display 160 in which multiple views of the patient anatomy are shown (e.g., external, coronal, sagittal, etc.). Indicia may be provided in one or more of the views to facilitate the surgeon understanding the location of the cutting tip 72, and more particularly the tracked point, relative to the patient anatomy. The indicia may include crosshairs, axes lines, or the like.

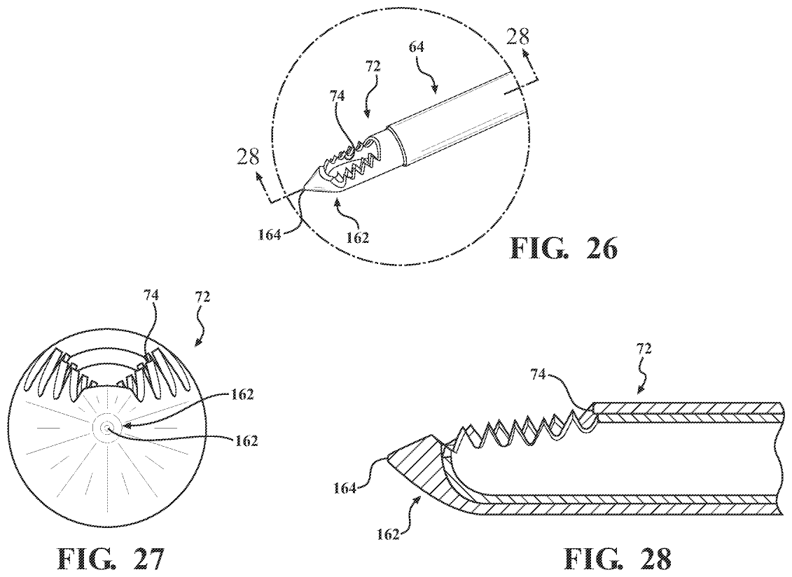

For conventional microdebrider/shavers, the rounded geometry of the cutting tip may result in distorted perception of the depictions of the instrument being displayed on the display. Referring to FIGS. 26-31, the cutting tip 72 of the cutting accessory 44 overcomes such shortcomings by providing tip geometry 162 has a tracked point 164 that is more accurately trackable, as well as intuitively understood by the surgeon viewing a displayed tracked point 164′ on the display 160. For instance, the symbol can contain a circle that represents the cutting window 74. In addition to the displayed tracked point 164′, additional visual indicia symbols can be provided based on the other aspects of the cutting tip 72. Therefore, in certain implementations, the navigation system 154 is configured to allow the surgeon to select a point or feature on the cutting tip visualized on the display 160, perhaps in addition to the tracked point. As shown in FIG. 25, the user may choose to visualize one or many of the features, including but not limited to a central axis of the tube assembly 64 (top), a midpoint of the cutting window 74 (upper middle), the opening of the cutting window 74 (lower middle), and a distalmost point of the cutting tip 72 (bottom).

As mentioned, the calibration data for navigation and tracking is associated with a well-defined point or feature on the cutting tip that is fixed relative to the cutting feature, such as a center of the cutting window, or a hood extension adjacent to a bur head. Such an association increases the accuracy and precision in calibration and tracking. With the tracked point being understood by the surgeon, uncertainty as to the location of the cutting window or the bur head may be eliminated. Known systems require the surgeon to use two devices: a dedicated pointer instrument and the cutting instrument. Here, however, the cutting tip may also be used as a “pointer” during procedures, for example, ENT procedures, such that the surgeon understands the pose (i.e., position and orientation) of the cutting tip being displayed in the navigation software. Since the defined feature is fixed or static on the cutting tip, additional indicia (e.g., graphical symbols) may be used on the display to provide additional information about the cutting tip, such as a location of the distal tip, center of the cutting window, edge of the cutting window, a longitudinal axis, and/or the like. The indicia may be rendered to more accurately resemble the cutting tip that may be unique to one of several selectable cutting accessories. In implementations in which the cutting accessory 44 is a shaver/microdebrider, the drive shaft 68 defines the suction lumen, and the feature is a distal point or center point of the cutting window 74. In implementations, in which the cutting tip 72 is a bur head, the feature may be a distal point or a center point of the bur head. The sensor 84 (e.g., the coil sensor) is spaced proximally from the feature by a calibrated distance.

The tip geometry 162 tip may extend directly from the end of the cutting tip 72 through its central axis, or it may extend along alternate vectors. The tip geometry 162 may form a distal pointed tip or geometry, as shown, or a ball geometry (e.g., a small sphere coupled to a distal end of the cutting tip 72). The pointed geometry may extend appreciably beyond the cutting window 74 by 0.5, 1.0, or 2.0 or more centimeters, for example. Variations in geometries are contemplated. The pointed tip described above may be manufactured through metal injection molding, machining, drawing, or other suitable fabrication processes. In one variant, the tip geometry 162 may take the form of extruded/embossed surfaces or features near the distal tip to communicate the tracked point 164 to the user. Additionally, or alternatively, decals using laser marking or etching may be used to indicate the tracked point 164 by the navigation system 154.

Aspects of the present disclosure are configured to be used on a cutting accessory 44 that is a bur. The bur may be a diamond bur, fluted bur, or any other suitable type and size of bur head. It is also contemplated that the aspect of the present disclosure may be used with other cutting implements such as a curette, rasp, blade tip, trephine, brush, or the like, or non-cutting manual or powered instruments such as a screwdriver, endoscopic camera, light assembly, or the like. Referring to FIGS. 18-22, the cutting tip 72 of the cutting accessory 44 includes a bur head, and the tip geometry 162 includes a hood extension from the outer tube 66. The hood extension may extend laterally adjacent to the bur head, in particular it may extend above and beyond the bur head as illustrated. The tracked point 164 of the tip geometry 162 may be a pointed tip of the hood extension. Like the pointed geometry of the microdebrider/shaver, the pointed tip of the hood extension provides a specific feature of the cutting tip 72 to the surgeon of the location being tracked. Therefore, when visualizing the indicia being displayed on the display 160, the surgeon readily appreciates the pose of the bur head relative to the adjacent anatomy. Further, as the tracked point 164 is an extension of the outer tube 66 (fixed or malleable), any relative movement of the cutting tip does not impact the point being tracked. Consequently, the cutting accessory 44 including the bur head may be implemented on a tube assembly 64 that is fixed, articulable, or malleable. Still further, the hood extension is configured to protect the tissue from the opposite side of the bur head. In one variant, the intermediate tube 70 may define a tubular distal end from which the bur head extends, and the hood extension may be coupled to the intermediate tube 70. Therefore, the hood extension may be operably coupled to an actuator, such that the surgeon may rotate the hood extension about the longitudinal axis as desired.

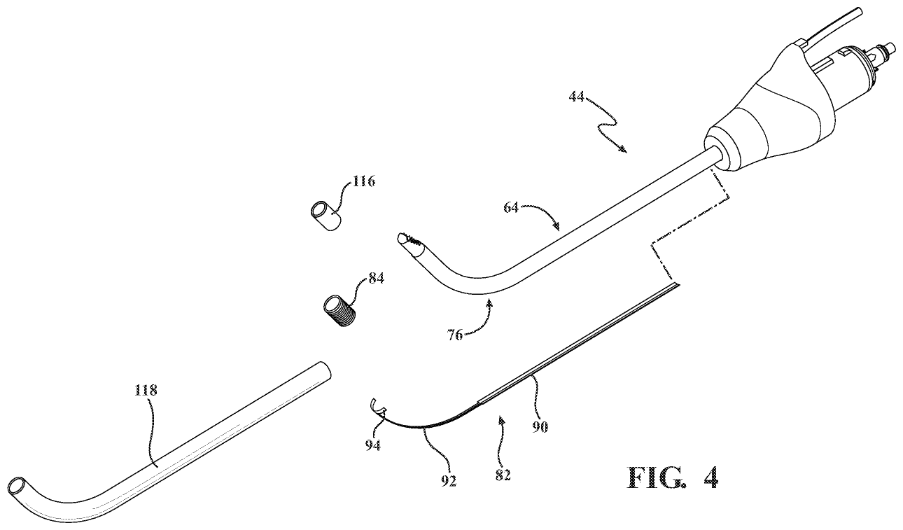

In any of the aforementioned implementations, the cutting accessory 44 provides for selective rotation of the cutting window 74 in an intuitive and ergonomic manner. Referring to FIGS. 13, 15, 16 and 21, the housing 126 may be formed of housing sections 170, 172 coupled to one another. The housing sections 170, 172 include several internal geometries defining voids and are configured to accommodate and support internal subcomponents of the cutting accessory 44. The housing 126 may include a base portion 184, and a neck 186 formed by contoured surfaces extending upwardly from the base portion 184. The contoured surfaces may function as support points of the cutting accessory 44 to be pinched between the index or middle finger and the thumb of the surgeon. The neck 186 terminates at a crown 188 defining a generally circular edge or opening. The crown 188 may be inclined relative to the longitudinal axis (LA) of the tube assembly 64, as best shown in FIGS. 4 and 9.

An actuator 190 is operably coupled to the housing 126. In the exemplary illustrated implementation, the actuator 190 is a dial or wheel positioned atop the crown 188 of the housing 126. An upper edge of the dial may be radiused or otherwise curved. The dial may include a lower edge approximating a shape of the crown 188 such that the contour of the neck 186 and crown 188 is generally continuous with the contour of the dial. Consequently, the surgeon may comfortably move digit positioning between the dial and the contoured surfaces of the housing 126. The dial may include ridges, texturizing, or other gripping features configured to provide tactile feedback to the surgeon with digit repositioning. Additionally, or alternatively, the dial may be sized slightly larger than the crown 188 to provide the surgeon with a starker tactile feel with digit repositioning. The dial may be coaxially aligned with a dial axis (A_{D}). The dial axis may be perpendicular to the inclination of the crown 188. An angle, a, defined between the dial axis and the longitudinal axis, may be an acute angle less than (and not equal to) 90 degrees. With the lower edge of the dial situated adjacent the crown 188, the dial is oriented distally upward. The orientation has been shown to be ergonomically superior to known devices in which a larger disc-like dial is oriented horizontally and requires support on an upper side of the disc-like dial. The implementation permits the dial to be supported from below and within the housing 126, which lessens visual obstruction during use of the surgical cutting instrument 40, as well as provides a flattened upper resting surface for the surgeon's index finger for ease with repeated and/or fine manipulation of the dial.

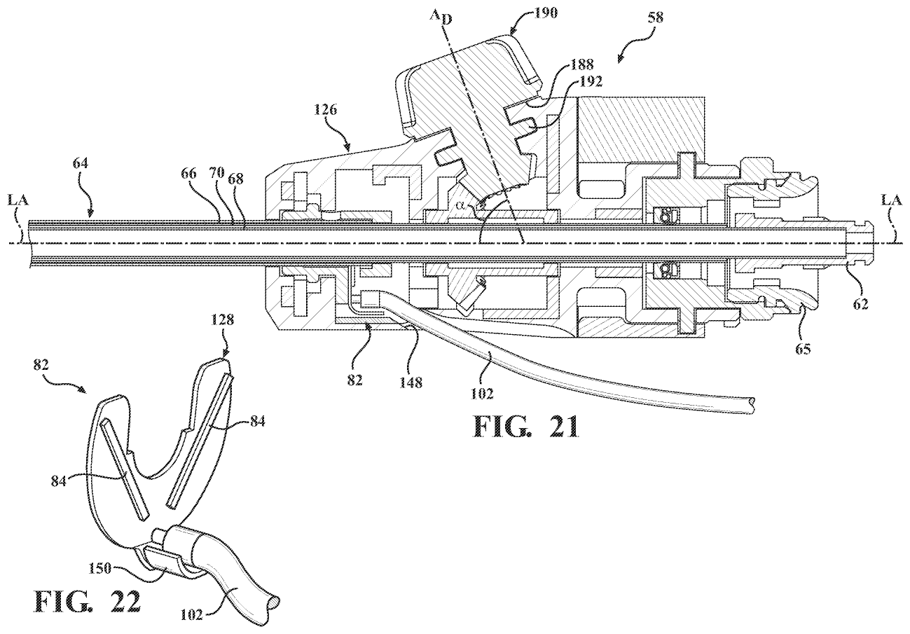

The actuator 190 includes a flange 192 rotatably encased within a void of the housing 126. As appreciated from FIGS. 15 and 21, the arrangement fixes the dial relative to the housing 126 in five degrees of freedom, but otherwise permits rotation of the dial about the dial axis. The dial further includes a lower bevel gear 194. The actuator 190 of the cutting accessory 44 includes an inner hub 196 with a complementary bevel gear 197 configured to engage the lower bevel gear 194 of the dial. The angled bevel gear may include a gear ratio within the range of approximately 0.5:1 to 3:1, or more particularly within the range of approximately 1:1 to 2:1. The inner hub 196 is rotatably supported by the internal geometries within the housing 126, and is fixedly coupled to the intermediate tube 70. As such, an input from the surgeon to rotate the dial imparts rotation of the intermediate tube 70 about the longitudinal axis. The cutting window 74, defined by the intermediate tube 70, rotates in a manner corresponding to the input to the dial.

The ability to rotate the cutting window 74 about the longitudinal axis may be provided on a tube assembly 64 that is rigid and straight, rigid and angled, articulable, bendable, or the like. In one variant to be further described in which the tube assembly is angled, articulable, or bendable, the actuator 190 (or a second, bend actuator) may be operably coupled to the outer tube 66. The actuator is configured to receive another input from the surgeon to alter the orientation of the bend (i.e., rotate a distal portion of the tube assembly 64 about the longitudinal axis). FIGS. 15 and 16 show a second inner hub 198 rotatably supported by the internal geometries within the housing 126. The second inner hub 198 is fixedly coupled to the outer tube 66. A collar 174 is operably coupled to the second inner hub 198 and supported within the housing 126. The second inner hub 198 and/or the collar 174 is configured to be coupled to the bend actuator (not shown) through an opening (not shown) of the housing 126. Another exemplary arrangement for selectively orienting the bend is disclosed in commonly owned International Publication No. WO 2022/224218, published Oct. 27, 2022, the entire contents of which are hereby incorporated by reference.

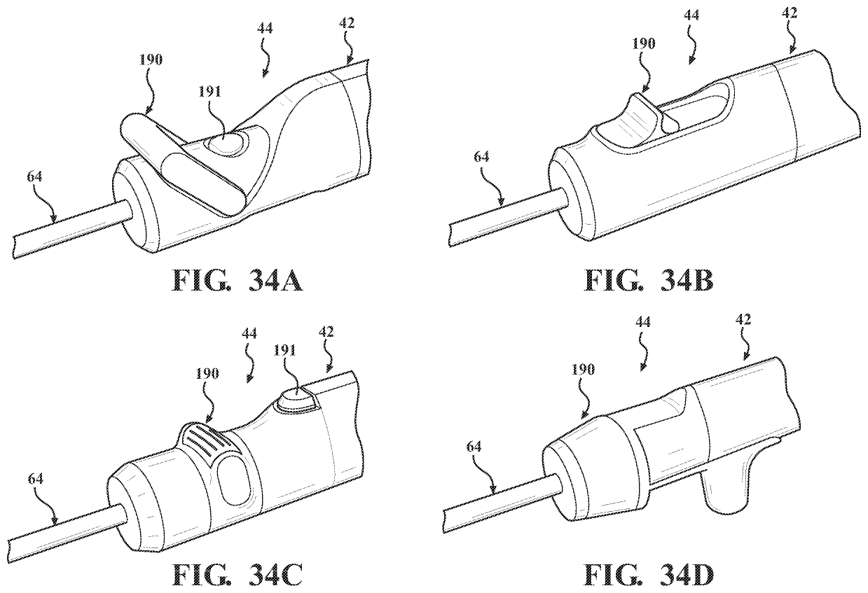

Alterative implementations of the actuator 190 for rotating the cutting window 74 are illustrated in FIGS. 34A-34D. FIG. 34A depicts a pivotable lever disposed within a recess of the housing 126. FIG. 34B depicts a slider movably disposed within a recess of the housing 126. The lever or the slider may be spring-biased to return from an actuated position to an original position in the absence of the input, or selectively positionable between the original and actuated positions. FIG. 34C depicts a barrel rotatably disposed within a recess of the housing 126. An axis about which the barrel is configured to be rotated may be oriented orthogonal to the longitudinal axis of the tube assembly 64. The barrel may be operably coupled to the intermediate tube (not shown) with a worm-gear arrangement. FIG. 34D depicts a wheel configured to be rotated about an axis that is coaxial with the longitudinal axis of the tube assembly 64. Further details of implementations including the wheel are disclosed in commonly owned United States Patent Publication No. 2020/0146702, published May 14, 2020, the entire contents of which are hereby incorporated by reference. The implementations of the actuator 190 include suitable gearing or other mechanisms to effectuate the rotation of the cutting window 74 while also maintaining sufficient clearance for the tube assembly 64 and other subcomponents disposed within the housing 126. The cutting accessory 44 may also include a release input 191 operably coupled to a latching mechanism (not shown) for disengaging the cutting accessory 44 from the handpiece 42. The release input 191 may be a button release, a slidable release, a press release, a pull release, or other means.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

Further inventive aspects of the present disclosure are represented in the following exemplary clauses:

Clause 1—A cutting accessory configured to be removably coupled to a handpiece of a surgical cutting instrument that includes a motor, and an electrical receptacle, the cutting accessory comprising: an outer hub configured to be operably coupled with the handpiece; a drive hub rotatably disposed within the outer hub and configured to be operably coupled to the motor; a tube assembly comprising an outer tube extending distally from the outer hub, an inner tube coupled to the drive hub and coaxially disposed within the outer tube, and a cutting tip; and a sensor assembly comprising at least one navigation sensor, an electrical bridge disposed within the outer hub and coupled to the at least one navigation sensor, and an electrical connector coupled to the outer hub and the electrical bridge, wherein the electrical connector is configured to be coupled to the electrical receptacle on the handpiece with the drive hub being operably coupled to the motor.

Clause 2—The cutting accessory of clause 1, wherein the outer hub comprises a proximally-directed surface, and wherein the electrical connector is disposed on the proximally-directed surface and positioned distal to the drive hub.

Clause 3—The cutting accessory of clause 1 or 2, wherein the electrical connector comprises six pins, wherein four of the six pins are configured to transmit navigation data, and two of the six pins are configured to transmit authentication data.

Clause 4—The cutting accessory of clause 1 or 2, wherein the electrical bridge comprises a board, a ribbon extending between and coupling the board and the electrical connector, and memory coupled to the board.

Clause 5—A cutting accessory configured to be removably coupled to a handpiece of a surgical cutting instrument that includes a motor, and an electrical receptacle, the cutting accessory comprising: an outer hub configured to be operably coupled with the handpiece and defining an opening; a drive hub rotatably disposed within the outer hub and configured to be operably coupled to the motor; a tube assembly comprising an outer tube extending distally from the outer hub, an inner tube coupled to the drive hub and coaxially disposed within the outer tube, and a cutting tip; and a sensor assembly comprising a board, at least one navigation sensor coupled to the board, and a sensor cable coupled to the board and in electrical communication with the at least one navigation sensor, wherein the sensor cable extends through the opening of the outer hub and configured to be coupled to complementary hardware of a navigation system.

Clause 6—The cutting accessory of clause 5, wherein the sensor assembly further comprises a flange extending from the board and supporting a junction between the sensor cable and the board.

Clause 7—The cutting accessory of any one of clauses 4-6, wherein the cutting tip comprises a tip geometry, wherein the memory stores calibration data indicative of a position of a tracked point of the tip geometry relative to the at least one navigation sensor.

Clause 8—The cutting accessory of clause 7, wherein the tip geometry is one of a pointed tip or a ball geometry.

Clause 9—The cutting accessory of any one of clauses 4-8, wherein the board defines a cutout or opening through which the outer tube and the inner tube extend.

Clause 10—The cutting accessory of clause 4, wherein the ribbon is arranged in a meandering configuration through voids defined by internal geometries within the outer hub.

Clause 11—The cutting accessory of clause 10, wherein the ribbon comprises at least two electrical traces that are tightly coupled and routed as a differential pair; and, optionally, wherein the traces have a width of less than 0.20 millimeters and a space separation between the traces is less than 0.25 millimeters.

Clause 12—The cutting accessory of any one of clauses 1-11, wherein the at least one navigation sensor is coupled to the outer tube adjacent to the cutting tip, wherein the cutting accessory further comprises at least one electrical trace extending along the outer tube and coupling the at least one navigation sensor to the board; and, optionally, wherein the at least one electrical trace is a twisted pair.

Clause 13—The cutting accessory of clause 12, further comprising a sheath coupled to an outer surface of the outer tube and overlying the at least one trace and the at least one navigation sensor.

Clause 14—The cutting accessory of clause 12, wherein the tube assembly further comprises an auxiliary tube extending from the outer hub and along an outer surface of the outer tube, wherein the at least one trace extends through the auxiliary tube.

Clause 15—The cutting accessory of any one of clauses 1-14, wherein the outer tube comprises a malleable region, and wherein the at least one navigation sensor is positioned distal to the malleable region.

Clause 16—A cutting accessory configured to be removably coupled to a handpiece of a surgical cutting instrument that includes a motor and an electrical receptacle, the cutting accessory comprising: an outer hub configured to be operably coupled with the handpiece; a drive hub rotatably disposed within the outer hub and configured to be operably coupled to the motor; a tube assembly comprising an outer tube extending distally from the outer hub, an inner tube coupled to the drive hub and coaxially disposed within the outer tube, and a cutting tip on the inner tube, wherein the outer tube comprises a malleable region, and wherein the inner tube comprises a flexible region disposed within the malleable region; and a sensor assembly comprising at least one navigation sensor coupled to the outer tube distal to the malleable region, and at least one trace coupled to the at least one navigation sensor, wherein the at least one trace extends along the outer tube to within the outer hub.

Clause 17—The cutting accessory of clause 16, wherein the malleable region comprises a series of slots that form a malleable spine configured to be bent and/or re-bent by a user to a shaped configuration, and maintain the tube assembly in the shaped configuration, wherein the at least one trace extends along the malleable spine.

Clause 18—The cutting accessory of clause 16 or 17, further comprising a sheath coupled to an outer surface of the outer tube and overlying the at least one trace and the at least one navigation sensor.

Clause 19—The cutting accessory of any one of clauses 16-18, wherein at least a portion of the at least one trace is arranged in a serpentine configuration.

Clause 20—The cutting accessory of any one of clauses 16-19, wherein the sensor assembly further comprises an electrical bridge disposed within the outer hub, wherein the at least one trace is coupled to the electrical bridge.

Clause 21—The cutting accessory of any one of clauses 16-20, wherein the sensor assembly further comprises memory storing calibration data indicative of a location of the at least one navigation sensor relative to a tracked point of the cutting tip.

Clause 22—A cutting accessory configured to be removably coupled to a handpiece of a surgical cutting instrument including a motor and an electrical receptacle, the cutting accessory comprising: an outer hub configured to be operably coupled with the handpiece; a drive hub rotatably disposed within the outer hub and configured to be operably coupled to the motor; a tube assembly comprising an outer tube extending distally from the outer hub, an inner tube coupled to the drive hub and coaxially disposed within the outer tube, and a cutting tip; and a sensor assembly comprising at least one navigation sensor, an electrical bridge comprising a board to which the at least one navigation sensor is coupled, a ribbon extending from the board and comprising traces in electronic communication with the at least one navigation sensor, and memory coupled to the board and storing calibration data indicative of a location of the at least one navigation sensor relative to a tracked point on the cutting tip.

Clause 23—The cutting accessory of clause 22, wherein the board defines a cutout or opening through which the outer tube and the inner tube extend.

Clause 24—The cutting accessory of clause 22 or 23, wherein the traces are tightly coupled and routed as a differential pair; and, optionally, wherein the traces have a width of less than 0.20 millimeters and a space separation between the traces is less than 0.25 millimeters.

Clause 25—The cutting accessory of any one of clauses 22-24, wherein the cutting tip comprises a tip geometry comprising the tracked point; and, optionally, wherein the tip geometry is one of a pointed tip or a ball geometry.

Clause 26—The cutting accessory of any one of clauses 22-25, wherein the memory further stores identification data indicative of characteristics of a type of the cutting accessory, and/or authentication data.

Clause 27—A cutting accessory configured to be removably coupled to a handpiece of a surgical cutting instrument including a motor and an electrical receptacle, the cutting accessory comprising: an outer hub configured to be coupled with the handpiece; a drive hub rotatably disposed within the outer hub and configured to be operably coupled to the motor; a tube assembly comprising an outer tube extending distally from the outer hub, an intermediate tube coaxially disposed within the outer tube, an inner tube coupled to the drive hub and coaxially disposed within the intermediate tube, and a cutting tip, wherein the tube assembly comprises a bend; a bend actuator operably coupled to the outer tube and configured to receive a user input to rotate the outer tube relative to the outer hub to selectively rotate the bend of the tube assembly about a longitudinal axis; and a sensor assembly comprising at least one navigation sensor coupled to the cutting tip at an axial position distal to the bend, an electronics bridge comprising a board disposed within the outer hub and coupled to the outer tube, and a ribbon extending from the board, wherein the ribbon is configured to provide slack to permit the board to rotate within the outer hub with rotation of the outer tube in response to the bend actuator receiving the user input from a user.

Clause 28—The cutting accessory of clause 27, wherein the ribbon is arranged in a meandering configuration through voids defined by internal geometries within the outer hub.

Clause 29—The cutting accessory of clause 28, wherein the meandering configuration comprises at least one folded feature in which the ribbon is stacked upon itself, wherein the at least one folded feature is configured to fold or unfold in response to rotation of the board.

Clause 30—The cutting accessory of clause 28 or 29, wherein the meandering configuration comprises at least one U-shaped section.

Clause 31—The cutting accessory of any one of clauses 27-30, wherein the ribbon comprises at least two traces that are tightly coupled and routed as a differential pair; and, optionally, wherein the at least two traces have a width of less than 0.20 millimeters, and wherein a space separation between the at least two traces is less than 0.25 millimeters.

Clause 32—A cutting accessory configured to be removably coupled to a handpiece of a surgical cutting instrument including a motor and an electrical receptacle, the cutting accessory comprising: an outer hub configured to be operably coupled with the handpiece; a drive hub rotatably disposed within the outer hub and configured to be operably coupled to the motor; a tube assembly comprising an outer tube extending distally from the outer hub, an inner tube coupled to the drive hub and coaxially disposed within the outer tube, and a cutting tip disposed on the inner tube, wherein the cutting tip comprises a distal pointed tip; and a sensor assembly comprising at least one navigation sensor, and memory storing calibration data indicative of a location of the distal pointed tip relative to the at least one navigation sensor.

Clause 33—The cutting accessory of clause 32, wherein the outer tube defines a cutting window, and wherein the distal pointed tip is integrally formed on the outer tube and distal to the cutting window.

Clause 34—The cutting accessory of clauses 32 or 33, wherein the distal pointed tip is coaxial with a central longitudinal axis of a distal segment of the tube assembly.

Clause 35—The cutting accessory of clause 32 or 33, wherein the distal pointed tip is arranged on a vector angled relative to a central longitudinal axis of a distal segment of the tube assembly.

Clause 36—The cutting accessory of any one of clauses 32-35, wherein the distal pointed tip comprises a marking or etching indicative of a point to be displayed on a display of a navigation system.

Clause 37—The cutting accessory of clause 36, wherein the cutting tip is a bur head, wherein the outer tube comprises a hood extension extending laterally distal to the bur head, wherein the hood extension comprises the distal pointed tip.

Clause 38—The cutting accessory of any one of clauses 1-37, wherein the at least one navigation sensor are two five degree of freedom sensors positioned relative to one another at a predefined angle.

Clause 39—The cutting accessory of any one of clauses 1-37, wherein the at least one navigation sensor is a six degree of freedom sensor.

Clause 40—An instrument for use with a navigation system, the instrument accessory comprising: a hub; an outer tube extending from the hub; a tip disposed at a distal end of the outer tube; a sensor assembly configured to be arranged in electronic communication with the navigation system, wherein the sensor assembly comprises: a substrate coupled to and extending along the outer tube, wherein the substrate comprises a flexible region, and a proximal region between the flexible region and the hub; electrical traces extending along the substrate, wherein the electrical traces are arranged in a twisted pair configuration in the proximal region, and a linear configuration in the flexible region; and a coil sensor coaxially disposed about the outer tube between the flexible region and the tip, wherein the coil sensor is in electrical communication with the electrical traces, and wherein the coil sensor is configured to detect changes in electric field induced by the navigation system.

Clause 41—An instrument for use with a navigation system, the instrument accessory comprising: a hub; an outer tube extending from the hub; a tip disposed at a distal end of the outer tube; a sensor assembly configured to be arranged in electronic communication with the navigation system, wherein the sensor assembly comprises: a substrate coupled to and extending along the outer tube; electrical traces extending along the substrate; and a coil sensor comprising a conductive wire coaxially wound about the outer tube and spaced proximal from a feature of the tip by a calibrated distance, wherein the coil sensor is in electrical communication with the electrical traces, and wherein the coil sensor is configured to detect changes in an electric field induced by the navigation system; and a sheath coaxially disposed about the substrate and the coil sensor.

Clause 42—An instrument for use with a navigation system, the instrument accessory comprising: a hub; an outer tube extending from the hub; a tip disposed at a distal end of the outer tube; a drive shaft coaxially and rotatably disposed within the outer tube; a sensor assembly configured to be arranged in electronic communication with the navigation system, wherein the sensor assembly comprises: a substrate coupled to and extending along the outer tube; electrical traces extending the substrate; an insulative spacer coupled to the substrate and coaxially disposed about the outer tube; and a coil sensor coaxially disposed about the insulative spacer, wherein the coil sensor is in electrical communication with the electrical traces, and wherein the coil sensor is configured to detect changes in an electric field induced by the navigation system.

Clause 43—An instrument for use with a navigation system, the instrument accessory comprising: a hub; an outer tube extending from the hub and comprising a bend or a bendable region; a tip disposed at a distal end of the outer tube; a sensor assembly configured to be arranged in electronic communication with the navigation system, wherein the sensor assembly comprises: a substrate coupled to and extending along the outer tube, wherein the substrate comprises a proximal region and a flexible region, wherein a width of the flexible region is less than a width of the proximal region and at least a portion of the flexible region of the substrate is aligned with the bend or the bendable region of the outer tube; electrical traces extending along the substrate; and a coil sensor coaxially disposed about the outer tube between the flexible region and the cutting tip, wherein the coil sensor is in electrical communication with the electrical traces, and wherein the coil sensor is configured to detect changes in an electric field induced by the navigation system.

Clause 44—An instrument for use with a navigation system, the instrument accessory comprising: a hub; an outer tube extending from the hub; a tip disposed at a distal end of the outer tube; and a sensor assembly configured to be arranged in electronic communication with the navigation system, wherein the sensor assembly comprises a coil sensor coaxially disposed about the outer tube, wherein the coil sensor is formed from a gauge of wire and a number of windings to provide a sensor sensitivity that compensates for material properties of the outer tube.

Clause 45—The instrument of any one of clauses 40-44, wherein the instrument is any one of a curette, rasp, blade tip, trephine, brush, screwdriver, endoscopic camera, light assembly, and suction instrument.

1. A cutting accessory for a powered surgical instrument for use with a navigation system, the cutting accessory comprising:
a hub configured to be removably coupled to a handpiece of the powered surgical instrument;
an outer tube extending from the hub;
a drive shaft coaxially and rotatably disposed within the outer tube;
a cutting tip disposed at a distal end of the drive shaft;
a sensor assembly configured to be arranged in electronic communication with the navigation system, wherein the sensor assembly comprises:
a substrate coupled to and extending along the outer tube, wherein the substrate comprises a flexible region, and a proximal region between the flexible region and the hub;
electrical traces extending along the substrate, wherein the electrical traces are arranged in a twisted pair configuration in the proximal region, and a linear configuration in the flexible region; and
a coil sensor coaxially disposed about the outer tube between the flexible region and the cutting tip, wherein the coil sensor is in electrical communication with the electrical traces, and wherein the coil sensor is configured to detect changes in electric field induced by the navigation system.

2. The cutting accessory of claim 1, further comprising an insulative spacer coupled to the substrate and coaxially disposed about the outer tube, wherein the coil sensor is coaxially disposed about the insulative spacer.

3. The cutting accessory of claim 2, further comprising an insulative layer coaxially disposed about the coil sensor.

4. The cutting accessory of claim 1, wherein the substrate further comprises a distal ring extending from the flexible region and coaxially disposed about the outer tube, wherein the coil sensor is coaxially disposed about the distal ring.

5. The cutting accessory of claim 1, further comprising a sheath coaxially disposed about the substrate and the coil sensor.

6. The cutting accessory of claim 1, wherein the twisted pair configuration comprises each of a pair of the electrical traces being arranged in complementary linear segments on opposing upper and lower surfaces of the substrate, and the linear segments being separated by vias in which the each of the pair of the electrical traces is routed between the upper and lower surfaces.

7. (canceled)

8. The cutting accessory of claim 6, wherein the linear configuration comprises each of the pair of the electrical traces being arranged in a complementary linear pathway on the opposing upper and lower surfaces of the substrate.

9. The cutting accessory of claim 1, wherein the substrate is elongate, and wherein a width of the flexible region is narrower than a width the proximal region.

10. The cutting accessory of claim 9, wherein the substrate further comprises a distal region extending from the flexible region, wherein the cutting accessory further comprises coil pads disposed on the distal region and providing electrical communication between the electrical traces and ends of the coil sensor.

11. The cutting accessory of claim 10, wherein a width of the distal region is wider than the width of the flexible region;

12. (canceled)

13. The cutting accessory of claim 1, further comprising:
proximal pads disposed on the proximal region of the substrate; and
a cable comprising wires coupled to the proximal pads, wherein the cable is configured to be coupled to a console of the navigation system.

14. The cutting accessory of claim 1, wherein the coil sensor is spaced proximally from a feature of the cutting tip by a calibrated distance.

15. The cutting accessory of claim 14, wherein the drive shaft is a cutting tube defining a suction lumen and the cutting tip is a cutting window, and wherein the feature is a distal point or center point of the cutting window.

16. The cutting accessory of claim 1, wherein the cutting tip is a bur head, and wherein the feature is a distal point or a center point of the bur head.

17. The cutting accessory of claim 1, wherein the outer tube comprises a bend and the drive shaft is configured to transmit torque through the bend, and wherein at least a portion of an axial length of the flexible region of the substrate corresponds to the bend of the outer tube.

18. The cutting accessory of claim 1, wherein the outer tube comprises a bendable region formed from malleable material and the drive shaft is configured to transmit torque through the bendable region, and wherein at least a portion of an axial length of the flexible region of the substrate corresponds to the bendable region of the outer tube.

19. A cutting accessory for a powered surgical instrument for use with a navigation system, the cutting accessory comprising:
a hub configured to be removably coupled to a handpiece of the powered surgical instrument;
an outer tube extending from the hub;
a drive shaft coaxially and rotatably disposed within the outer tube;
a cutting tip disposed at a distal end of the drive shaft;
a sensor assembly configured to be arranged in electronic communication with the navigation system, wherein the sensor assembly comprises:
a substrate coupled to and extending along the outer tube;
electrical traces extending along the substrate; and
a coil sensor comprising a conductive wire coaxially wound about the outer tube and spaced proximal from a feature of the cutting tip by a calibrated distance, wherein the coil sensor is in electrical communication with the electrical traces, and wherein the coil sensor is configured to detect changes in an electric field induced by the navigation system; and
a sheath coaxially disposed about the substrate and the coil sensor.

20. The cutting accessory of claim 19, further comprising an insulative layer coaxially disposed about the coil sensor.

21. The cutting accessory of claim 19, wherein the substrate further comprises a distal ring coaxially disposed about the outer tube, wherein the coil sensor is coaxially disposed about the distal ring.

22. A cutting accessory for a powered surgical instrument for use with a navigation system, the cutting accessory comprising:
a hub configured to be removably coupled to a handpiece of the powered surgical instrument;
an outer tube extending from the hub;
a drive shaft coaxially and rotatably disposed within the outer tube;
a cutting tip disposed at a distal end of the drive shaft;
a sensor assembly configured to be arranged in electronic communication with the navigation system, wherein the sensor assembly comprises:
a substrate coupled to and extending along the outer tube;
electrical traces extending the substrate;
an insulative spacer coupled to the substrate and coaxially disposed about the outer tube; and
a coil sensor coaxially disposed about the insulative spacer, wherein the coil sensor is in electrical communication with the electrical traces, and wherein the coil sensor is configured to detect changes in an electric field induced by the navigation system.

23. The cutting accessory of claim 22, further comprising a sheath coaxially disposed about the substrate and the coil sensor; and, optionally, wherein the sheath is heat shrink material.

24. The cutting accessory of claim 23, wherein the substrate is radially disposed between the coil sensor and the sheath.

25-40. (canceled)
